(19) United States
(12) Patent Application Publication
(10) Pub. No.: US 2016/0022683 A1
(43) Pub. Date: Jan. 28, 2016

(54) COMBINATIONS OF BRUTON'S TYROSINE KINASE INHIBITORS AND CYP3A4 INHIBITORS

(71) Applicant: PHARMACYCLICS LLC, Sunnyvale, CA (US)

(72) Inventors: Maria FARDIS, San Carlos, CA (US); Juthamas SUKBUNTHERNG, Redwood City, CA (US)

(21) Appl. No.: 14/774,292

(22) PCT Filed: Mar. 12, 2014
(86) PCT No.: PCT/US14/24966

§ 371 Date: Sep. 10, 2015

Related U.S. Application Data
(60) Provisional application No. 61/784,119, filed on Mar. 14, 2013.

(51) Int. Cl. A61K 31/519; A61K 36/752; A61K 31/5377; A61K 31/426; A61K 45/06; A61K 31/496

(52) U.S. Cl. CPC A61K 31/519; A61K 45/06; A61K 31/496; A61K 31/5377; A61K 31/426; A61K 36/752

(57) ABSTRACT

Combinations of Bruton's tyrosine kinase (Btk) inhibitors, e.g., 1-((R)-3-(4-amino-3-(4-phenoxyphenyl)-1H-pyrazolo[3,4-d]pyrimidin-1-yl)piperidin-1-yl)prop-2-en-1-one, with CYP3A4 inhibitors are provided. Also provided are methods of treating cancers, and autoimmune disorders by administering combinations of Bruton's tyrosine kinase (Btk) inhibitors, e.g., 1-4((R)-3-(4-amino-3-(4-phenoxyphenyl)-1 H-pyrazolo[3, 4-d]pyrimidin-1-yl)piperidin-1-yl)prop-2-en-1-one, and CYP3A4 inhibitors.

### RELATED APPLICATIONS

This application claims the benefit of U.S. provisional patent application No. 61/784,119 entitled “COMBINATIONS OF BRUTON′S TYROSINE KINASE INHIBITORS AND CYP3A4 INHIBITORS” filed on Mar. 14, 2013, which is herein incorporated by reference in its entirety.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival. In addition, Btk plays a role in a number of other hematopoietic cell signaling pathways, e.g., Toll like receptor (TLR) and cytokine receptor-mediated TNF-α production in macrophages, IgE receptor signaling in Mast cells, inhibition of Fas/APO-1 apoptotic signaling in B-lineage lymphoid cells, and collagen-stimulated platelet aggregation.

1-((R)-3-(4-amino-3-(4-phenoxyphenyl)-1H-pyrazolo[3,4-d]pyrimidin-1-yl)-piperidin-1-yl)prop-2-en-1-one is also known by its IUPAC name as 1-{(3R)-3[4-amino-3-(4-phenoxyphenyl)-1H-pyrazolo[3,4d]pyrimidin-1-yl]piperidin-1yl-}prop-2-en-1one or 2-Propen-1-one, 1-[(3R)-3-[4-amino-3-(4-phenoxyphenyl)-1H-pyrazolo[3,4-d]pyrimidin-l-yl]-1-piperidinyl-, and has been given the USAN name, Ibrutinib. The various names given for Ibrutinib are used interchangeably herein.

### SUMMARY OF THE INVENTION

Disclosed herein, in certain embodiments, is a pharmaceutical composition comprising: (a) a therapeutically-effective amount of Ibrutinib; (b) a CYP3A4 inhibitor; and (c) a pharmaceutically-acceptable excipient. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the therapeutically-effective amount of Ibrutinib is between about 10 mg to about 100 mg. In some embodiments, the therapeutically-effective amount of Ibrutinib is between about 40 mg and about 100 mg. In some embodiments, the therapeutically-effective amount of Ibrutinib is between about 40 mg and about 70 mg. In some embodiments, the therapeutically-effective amount of Ibrutinib is about 40 mg. In some embodiments, the pharmaceutical composition is in a combined dosage form. In some embodiments, the pharmaceutical composition comprises an amount of the CYP3A4 inhibitor that is effective to increase the oral bioavailability of Ibrutinib. In some embodiments, the pharmaceutical composition comprises an amount of the CYP3A4 inhibitor that is effective to increase the Cmax of Ibrutinib. In some embodiments, the pharmaceutical composition comprises an amount of the CYP3A4 inhibitor that is effective to increase the Cmax of Ibrutinib by about 20× to about 40× the Cmax of Ibrutinib administered without a CYP3A4 inhibitor, or about 25× to about 35×. In some embodiments, the pharmaceutical composition comprises an amount of the CYP3A4 inhibitor that is effective to increase the AUC of Ibrutinib. In some embodiments, the pharmaceutical composition comprises an amount of the CYP3A4 inhibitor that is effective to increase the AUC of Ibrutinib by about 15× to about 35× the AUC of Ibrutinib administered without a CYP3A4 inhibitor, or about 20× to about 30×. In some embodiments, the pharmaceutical composition comprises an amount of the CYP3A4 inhibitor that is effective to increase the AUC of Ibrutinib by about 2× to about 35× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 30× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 25× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 20× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 15× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 10× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 5× the AUC of Ibrutinib administered without a CYP3A4 inhibitor. In some embodiments, the pharmaceutical composition comprises an amount of the CYP3A4 inhibitor that is effective to increase the AUC of Ibrutinib by about 2× to about 4× the AUC of Ibrutinib administered without a CYP3A4 inhibitor. In some embodiments, the composition does not significantly affect the Tmax or T1/2 of Ibrutinib as compared to the Tmax and T1/2 of Ibrutinib administered without a CYP3A4 inhibitor. In some embodiments, the pharmaceutical composition further comprises chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the pharmaceutical composition further comprises cyclophosphamide, hydroxydaunorubicin, vincristine, and prednisone, and optionally, rituximab. In some embodiments, the pharmaceutical composition further comprises bendamustine, and rituximab. In some embodiments, the pharmaceutical composition further comprises fludarabine, cyclophosphamide, and rituximab. In some embodiments, the pharmaceutical composition further comprises cyclophosphamide, vincristine, and prednisone, and optionally, rituximab. In some embodiments, the pharmaceutical composition further comprises etoposide, doxorubicin, vincristine, cyclophosphamide, prednisolone, and optionally, rituximab. In some embodiments, the pharmaceutical composition further comprises dexamethasone and lenalidomide.

Disclosed herein, in certain embodiments, is a pharmaceutical combination comprising a therapeutically-effective amount of Ibrutinib and a CYP3A4 inhibitor. In some embodiment, the combination is in a combined dosage form. In some embodiment, the combination is in separate dosage forms. In some embodiments, the Ibrutinib and the CYP3A4 inhibitor are administered concurrently. In some embodiments, the Ibrutinib and the CYP3A4 inhibitor are administered simultaneously, essentially simultaneously or within the same treatment protocol. In some embodiments, the Ibrutinib and the CYP3A4 inhibitor are administered sequentially. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; troleandromycin; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiment, the therapeutically-effective amount of Ibrutinib is between about 10 mg to about 100 mg. In some embodiments, the therapeutically-effective amount of Ibrutinib is between about 40 mg and about 100 mg. In some embodiments, the therapeutically-effective amount of Ibrutinib is between about 40 mg and about 70 mg. In some embodiments, the therapeutically-effective amount of Ibrutinib is about 40 mg. In some embodiments, the pharmaceutical combination comprises an amount of the CYP3A4 inhibitor that is effective to increase the oral bioavailability of Ibrutinib. In some embodiments, the pharmaceutical combination comprises an amount of the CYP3A4 inhibitor that is effective to increase the Cmax of Ibrutinib. In some embodiments, the pharmaceutical combination comprises an amount of the CYP3A4 inhibitor that is effective to increase the Cmax of Ibrutinib by about 20× to about 40× the Cmax of Ibrutinib administered without a CYP3A4 inhibitor, or about 25× to about 35×. In some embodiments, the pharmaceutical combination comprises an amount of the CYP3A4 inhibitor that is effective to increase the AUC of Ibrutinib. In some embodiments, the pharmaceutical combination comprises an amount of the CYP3A4 inhibitor that is effective to increase the AUC of Ibrutinib by about 15× to about 35× the AUC of Ibrutinib administered without a CYP3A4 inhibitor, or about 20× to about 30×. In some embodiments, the pharmaceutical combination comprises an amount of the CYP3A4 inhibitor that is effective to increase the AUC of Ibrutinib by about 2× to about 30× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 25× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 20× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 15× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 10× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 5× the AUC of Ibrutinib administered without a CYP3A4 inhibitor. In some embodiments, the pharmaceutical combination comprises an amount of the CYP3A4 inhibitor that is effective to increase the AUC of Ibrutinib by about 2× to about 4× the AUC of Ibrutinib administered without a CYP3A4 inhibitor. In some embodiments, the pharmaceutical combination does not significantly affect the Tmax or T1/2 of Ibrutinib as compared to the Tmax and T1/2 of Ibrutinib administered without a CYP3A4 inhibitor. In some embodiments, the pharmaceutical combination further comprises chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the pharmaceutical combination further comprises cyclophosphamide, hydroxydaunorubicin, vincristine, and prednisone, and optionally, rituximab. In some embodiments, the pharmaceutical combination further comprises bendamustine, and rituximab. In some embodiments, the pharmaceutical combination further comprises fludarabine, cyclophosphamide, and rituximab. In some embodiments, the pharmaceutical combination further comprises cyclophosphamide, vincristine, and prednisone, and optionally, rituximab. In some embodiments, the pharmaceutical combination further comprises etoposide, doxorubicin, vincristine, cyclophosphamide, prednisolone, and optionally, rituximab. In some embodiments, the pharmaceutical combination further comprises dexamethasone and lenalidomide.

Disclosed herein, in certain embodiments, is a method of treating a B-cell proliferative disorder in an individual in need thereof comprising administering a combination of: (a) a therapeutically-effective amount Ibrutinib; and (b) a CYP3A4 inhibitor. In some embodiments, the B-cell proliferative disorder is chronic lymphocytic leukemia (CLL), small lymphocytic lymphoma (SLL), high risk CLL, or a non-CLL/SLL lymphoma. In some embodiments, the B-cell proliferative disorder is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the B-cell proliferative disorder is acute or chronic myelogenous (or myeloid) leukemia, myelodysplastic syndrome, or acute lymphoblastic leukemia. In some embodiments, the B-cell proliferative disorder is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the B-cell proliferative disorder is high risk CLL or high risk SLL. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the therapeutically-effective amount of Ibrutinib is between about 10 mg to about 100 mg. In some embodiments, the therapeutically-effective amount of Ibrutinib is between about 40 mg and about 100 mg. In some embodiments, the therapeutically-effective amount of Ibrutinib is between about 40 mg and about 70 mg. In some embodiments, the therapeutically-effective amount of Ibrutinib is about 40 mg. In some embodiments, the method comprises an amount of the CYP3A4 inhibitor that is effective to increase the oral bioavailability of Ibrutinib. In some embodiments, the method comprises an amount of the CYP3A4 inhibitor that is effective to increase the Cmax of Ibrutinib. In some embodiments, the method comprises an amount of the CYP3A4 inhibitor that is effective to increase the Cmax of Ibrutinib by about 20× to about 40× the Cmax of Ibrutinib administered without a CYP3A4 inhibitor, or about 25× to about 35×. In some embodiments, the method comprises an amount of the CYP3A4 inhibitor that is effective to increase the AUC of Ibrutinib. In some embodiments, the method comprises an amount of the CYP3A4 inhibitor that is effective to increase the AUC of Ibrutinib by about 15× to about 35× the AUC of Ibrutinib administered without a CYP3A4 inhibitor, or about 20× to about 30×. In some embodiments, the method comprises an amount of the CYP3A4 inhibitor that is effective to increase the AUC of Ibrutinib by about 2× to about 35× the AUC of Ibrutinib administered without a CYP3A4 inhibitor. In some embodiments, the method comprises an amount of the CYP3A4 inhibitor that is effective to increase the AUC of Ibrutinib by about 2× to about 30× the AUC of Ibrutinib administered without a CYP3A4 inhibitor. In some embodiments, the method comprises an amount of the CYP3A4 inhibitor that is effective to increase the AUC of Ibrutinib by about 2× to about 25× the AUC of Ibrutinib administered without a CYP3A4 inhibitor. In some embodiments, the method comprises an amount of the CYP3A4 inhibitor that is effective to increase the AUC of Ibrutinib by about 2× to about 20× the AUC of Ibrutinib administered without a CYP3A4 inhibitor. In some embodiments, the method comprises an amount of the CYP3A4 inhibitor that is effective to increase the AUC of Ibrutinib by about 2× to about 15× the AUC of Ibrutinib administered without a CYP3A4 inhibitor. In some embodiments, the method comprises an amount of the CYP3A4 inhibitor that is effective to increase the AUC of Ibrutinib by about 2× to about 10× the AUC of Ibrutinib administered without a CYP3A4 inhibitor. In some embodiments, the method comprises an amount of the CYP3A4 inhibitor that is effective to increase the AUC of Ibrutinib by about 2× to about 5× the AUC of Ibrutinib administered without a CYP3A4 inhibitor. In some embodiments, the method comprises an amount of the CYP3A4 inhibitor that is effective to increase the AUC of Ibrutinib by about 2× to about 4× AUC of Ibrutinib administered without a CYP3A4 inhibitor. In some embodiments, the method comprises the pharmaceutical combination does not significantly affect the Tmax or T1/2 of Ibrutinib as compared to the Tmax and T1/2 of Ibrutinib administered without a CYP3A4 inhibitor. In some embodiments, the Ibrutinib and the CYP3A4 inhibitor are in a combined dosage form. In some embodiments, the Ibrutinib and the CYP3A4 inhibitor are in separate dosage forms. In some embodiments, the Ibrutinib and the CYP3A4 inhibitor are administered concurrently. In some embodiments, the Ibrutinib and the CYP3A4 inhibitor are administered simultaneously, essentially simultaneously or within the same treatment protocol. In some embodiments, the Ibrutinib and the CYP3A4 inhibitor are administered sequentially. In some embodiments, the method further comprises co-administering chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the method further comprises co-administering cyclophosphamide, hydroxydaunorubicin, vincristine, and prednisone, and optionally, rituximab. In some embodiments, the method further comprises co-administering bendamustine, and rituximab. In some embodiments, the method further comprises co-administering fludarabine, cyclophosphamide, and rituximab. In some embodiments, the method further comprises co-administering cyclophosphamide, vincristine, and prednisone, and optionally, rituximab. In some embodiments, the method further comprises co-administering etoposide, doxorubicin, vincristine, cyclophosphamide, prednisolone, and optionally, rituximab. In some embodiments, the method further comprises co-administering dexamethasone and lenalidomide.

### BRIEF DESCRIPTION OF THE FIGURES

FIG. 1. Illustrates a 72 hour time profile of mean plasma concentration of Ibrutinib when Ibrutinib is administered alone (Day 1) or in combination with ketoconazole, a CYP3A4 inhibitor (Day 7).

FIG. 2. Illustrates a 24 hour time profile of mean plasma concentration of Ibrutinib when Ibrutinib is administered alone (Day 1) or in combination with ketoconazole, a CYP3A4 inhibitor (Day 7).

FIG. 3. Illustrates a 72 hour time profile of mean plasma concentration of PCI-45227, a metabolite of Ibrutinib, when Ibrutinib is administered alone (Day 1) or in combination with ketoconazole, a CYP3A4 inhibitor (Day 7).

FIG. 4. Illustrates a 24 hour time profile of mean plasma concentration of PCI-45227 when Ibrutinib is administered alone (Day 1) or in combination with ketoconazole, a CYP3A4 inhibitor (Day 7).

FIG. 5. Illustrates the dose normalized Cmax of Ibrutinib by treatment and subject.

FIG. 6. Illustrates the dose normalized Cmax of PCI-45227 by treatment and subject.

FIG. 7. Illustrates the dose normalized AUClast of Ibrutinib by treatment and subject.

FIG. 8. Illustrates the dose normalized AUClast of PCI-45227 by treatment and subject.

FIG. 9. Illustrates a 24 hour time profile of mean plasma concentration of Ibrutinib when Ibrutinib is administered in the fed state, alone or in combination with grapefruit juice, a CYP3A4 inhibitor.

FIG. 10. Illustrates a 24 hour time profile of mean plasma concentration of Ibrutinib when Ibrutinib is administered alone (Day 1) or in combination with rifampin, a CYP3A4 inducer (Day 11).

FIG. 11. Illustrates the change in AUC versus baseline apparent clearance following oral administration of Ibrutinib with ketoconazole, grapefruit juice, and rifampin.

### DETAILED DESCRIPTION OF THE INVENTION

Small molecule Btk inhibitors, such as Ibrutinib, are useful for reducing the risk of or treating a variety of diseases affected by or affecting many cell types of the hematopoietic lineage including, e.g., autoimmune diseases, heteroimmune conditions or diseases, inflammatory diseases, cancer (e.g., B-cell proliferative disorders), and thromboembolic disorders.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Bioavailability” refers to the percentage of Ibrutinib dosed that is delivered into the general circulation of the animal or human being studied. The total exposure (AUC(0-∝)) of a drug when administered intravenously is usually defined as 100% bioavailable (F %). “Oral bioavailability” refers to the extent to which Ibrutinib is absorbed into the general circulation when the pharmaceutical composition is taken orally as compared to intravenous injection.

“Blood plasma concentration” refers to the concentration of Ibrutinib in the plasma component of blood of a subject. It is understood that the plasma concentration of Ibrutinib may vary significantly between subjects, due to variability with respect to metabolism and/or possible interactions with other therapeutic agents. In accordance with one embodiment disclosed herein, the blood or plasma concentration of Ibrutinib may vary from subject to subject. Likewise, values such as maximum plasma concentration (Cmax) or time to reach maximum plasma concentration (Tmax), or total area under the plasma concentration time curve (AUC(0-∝)) may vary from subject to subject. Due to this variability, the amount necessary to constitute “a therapeutically effective amount” of Ibrutinib may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Ibrutinib, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The terms “subject”, “patient” and “individual” are used interchangeably. As used herein, they refer to an animal. By way of example only, a subject may be, but is not limited to, a mammal including, but not limited to, a human. The terms do not require the supervision (whether continuous or intermittent) of a medical professional.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC50 refers to an amount, concentration or dosage of a particular test compound that achieves a 50% inhibition of a maximal response, such as inhibition of Btk, in an assay that measures such response.

As used herein, EC50 refers to a dosage, concentration or amount of a particular test compound that elicits a dose-dependent response at 50% of maximal expression of a particular response that is induced, provoked or potentiated by the particular test compound.

### Btk Inhibitor Compounds Including Ibrutinib, and Pharmaceutically Acceptable Salts Thereof

The Btk inhibitor compounds described herein are selective for Btk and kinases having a cysteine residue in an amino acid sequence position of the tyrosine kinase that is homologous to the amino acid sequence position of cysteine 481 in Btk. The Btk inhibitor compounds can form a covalent bond with Cys 481 of Btk (e.g., via a Michael reaction).

In some embodiments, the Btk inhibitor is AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37, (Ono Pharmaceutical Co., Ltd.), PLS-123 (Peking University), RN486 (Hoffmann-La Roche), or HM71224 (Hanmi Pharmaceutical Company Limited).

In some embodiments, the Btk inhibitor is 4-(tert-butyl)-N-(2-methyl-3-(4-methyl-6-4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)-N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxmethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-l-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-y1)amino)methyl)benzamide (HY11066).

In some embodiments, the Btk inhibitor is:

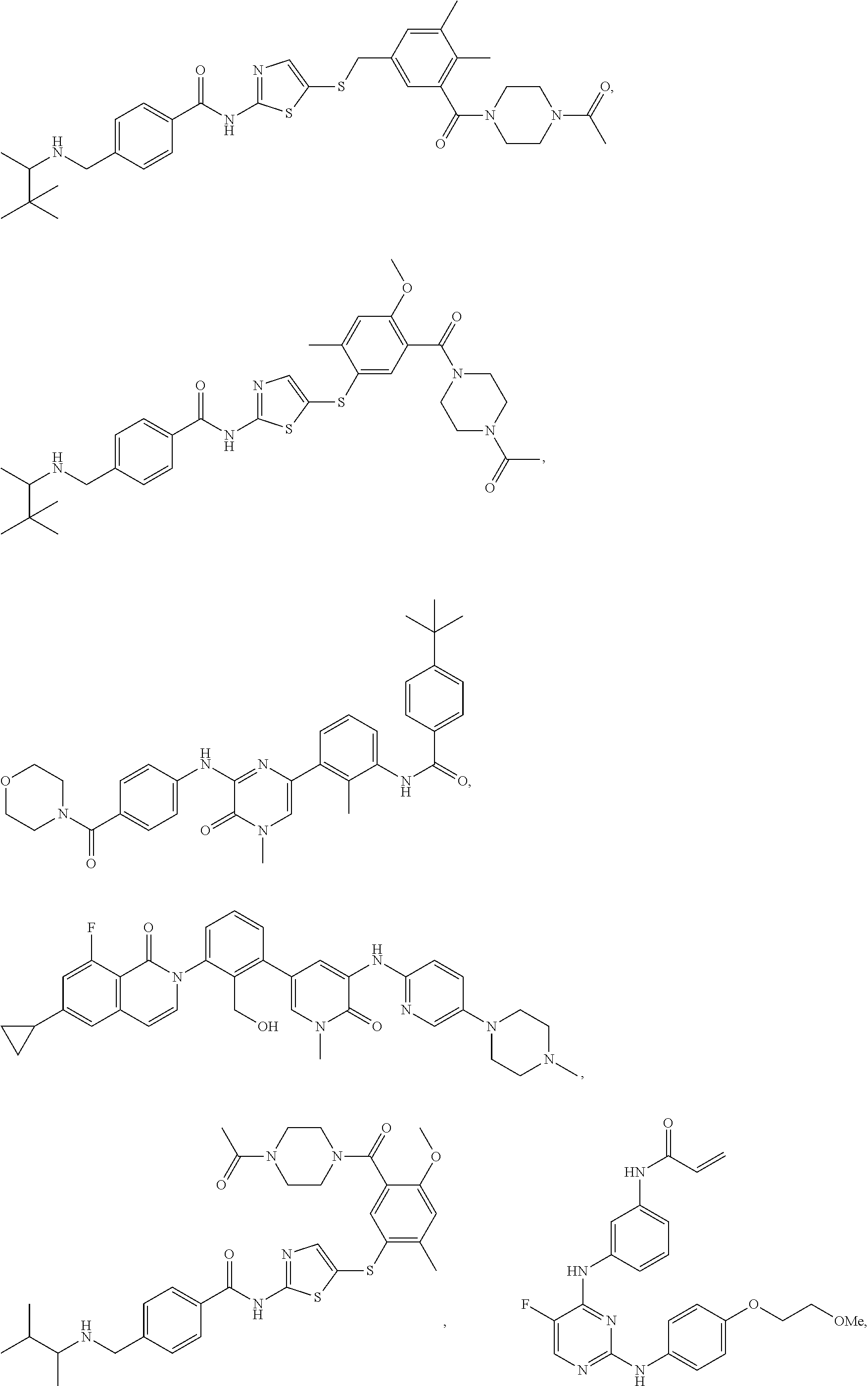

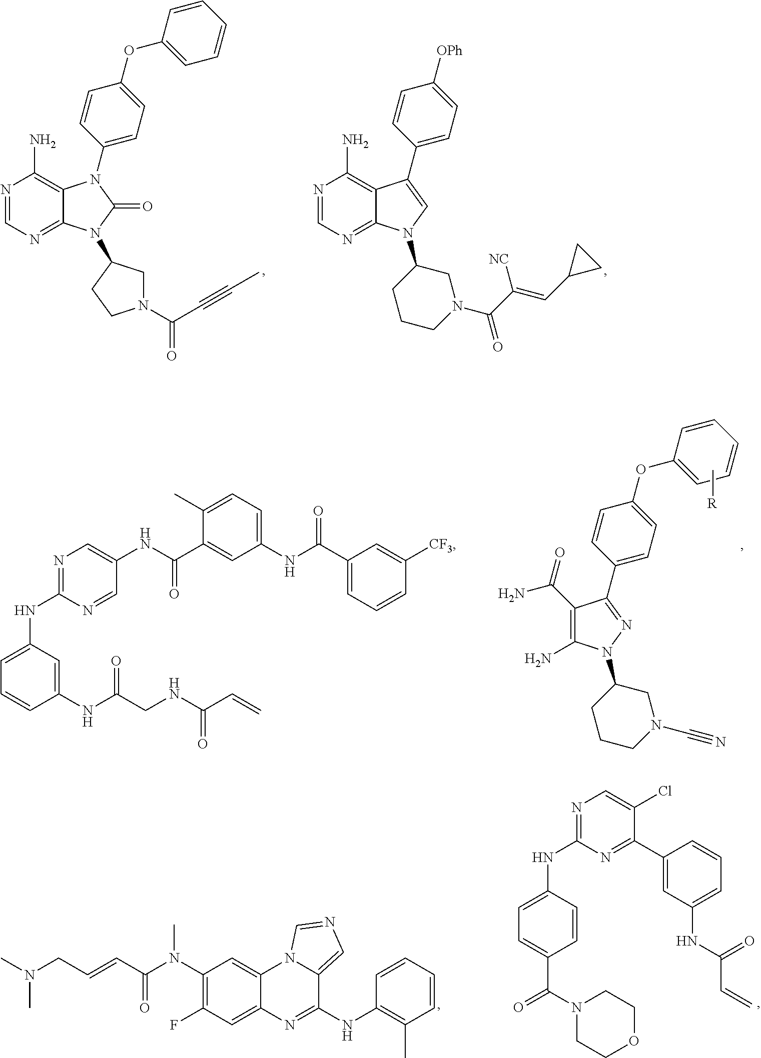

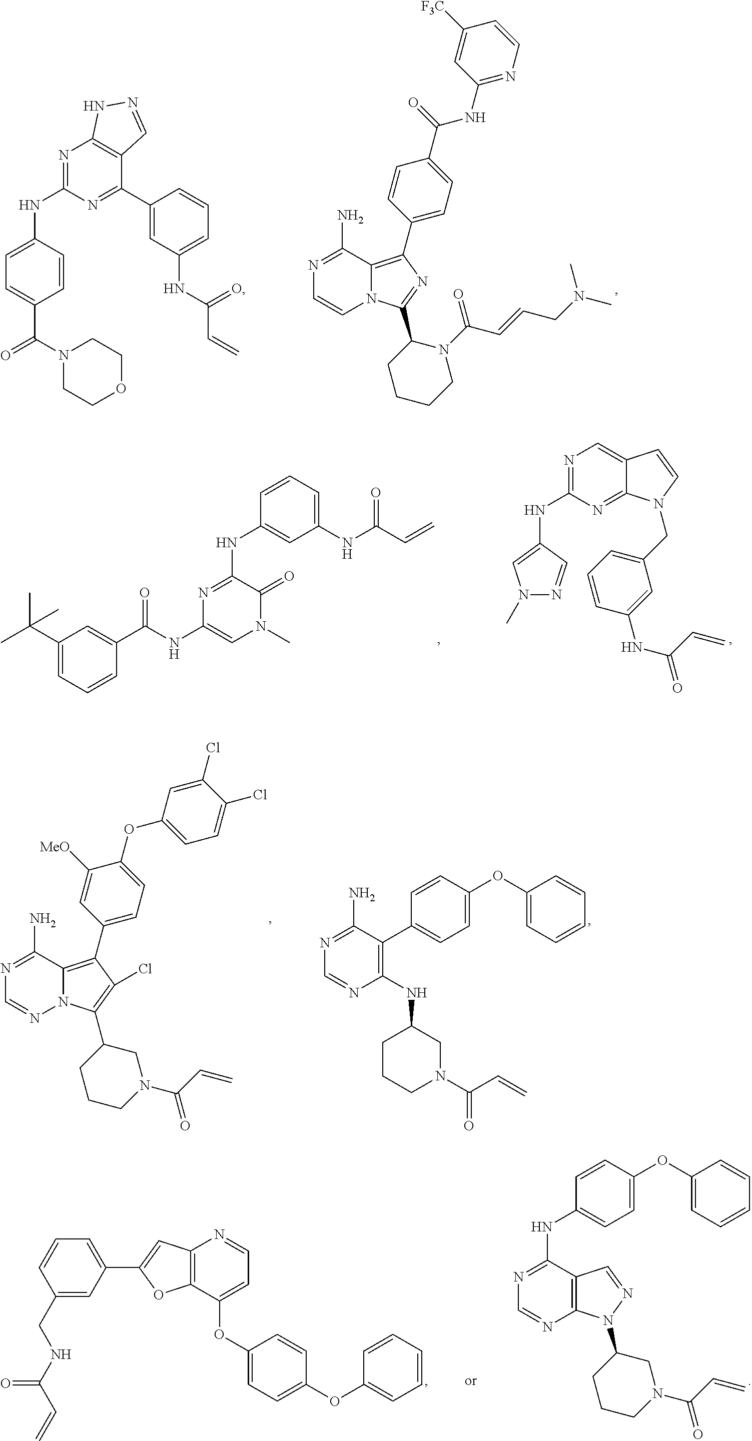

In some embodiments, the Btk inhibitor is Ibrutinib. “Ibrutinib” or “1-((R)-3-(4-amino-3-(4-phenoxyphenyl)-1H-pyrazolo[3 ,4-d]pyrimidin-1-yl)piperidin-1 -yl)prop-2-en-1-one” or “1-{(3R)-3-[4-amino-3-(4-phenoxyphenyl)-1H-pyrazolo [3 ,4-d]pyrimidin-1-y1]piperidin-1-yl}prop-2-en-1-one” or “2-Propcn-1-one, 1-[(3R)-3-[4-amino-3 -(4-phenoxyphenyl)-1H-pyrazolo [3,4-d]pyrimidin-1-yl9 -1-piperidinyl-” or Ibrutinib or any other suitable name refers to the compound with the following structure:

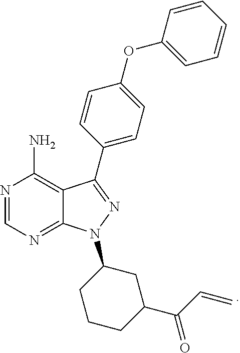 [SMILES: C=CC(=O)C1CCC[C@@H](n2nc(-c3ccc(Oc4ccccc4)cc3)c3c(N)ncnc32)C1]

PCI-45227, a metabolite of Ibrutinib, refers to 1-(R)-3-(4-amino-3-(4-phenoxyphenyl)-1H-pyrazolo[3,4-d]pyrimidin-1-yl)piperidin-1-yl)-2,3-dihydroxypropan-1-one.

A wide variety of pharmaceutically acceptable salts is formed from Ibrutinib and includes:

acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;

acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

Combination with CYP3A Inhibitors

Disclosed herein, in certain embodiments, are pharmaceutical combinations comprising a Btk inhibitor compound and a CYP3A inhibitor.

Further disclosed herein, in certain embodiments, are pharmaceutical combinations comprising Ibrutinib and a CYP3A inhibitor.

Cytochrome P450 3A (abbreviated CYP3A), is a member of the cytochrome P450 mixed-function oxidase system. The CYP3A locus includes all the known members of the 3A subfamily of the cytochrome P450 superfamily of genes. These genes encode monooxygenases which catalyze many reactions involved in drug metabolism and synthesis of cholesterol, steroids and other lipids. The CYP3A cluster consists of four genes; CYP3A4, CYP3A5, CYP3A7, and CYP3A43.

Cytochrome P450 enzymes modify a variety of substrate, including hydroxylation, epoxidation, aromatic oxidations, heteroatom oxidations, N- and O-dealkylations, aldehyde oxidations, and dehydrogenations.

In some embodiments, Ibrutinib and a CYP3A inhibitor are co-administration concurrently (e.g., simultaneously, essentially simultaneously or within the same treatment protocol) or sequentially.

In some embodiments, Ibrutinib and a CYP3A inhibitor are co-administered in separate dosage forms. In some embodiments, Ibrutinib and a CYP3A inhibitor are co-administered in combined dosage forms.

In some embodiments, the co-administration of Ibrutinib and a CYP3A inhibitor increases the oral bioavailability of Ibrutinib. In some embodiments, the co-administration of Ibrutinib and a CYP3A inhibitor increases the Cmax of Ibrutinib. In some embodiments, the co-administration of Ibrutinib and a CYP3A inhibitor increases the AUC of Ibrutinib.

Disclosed herein, in some embodiments, the CYP3A inhibitor is a CYP3A4 inhibitor. In some embodiments, the CYP3A inhibitor is a CYP3A5 inhibitor. In some embodiments, the CYP3A inhibitor is a CYP3A7 inhibitor. In some embodiments, the CYP3A inhibitor is a CYP3A43 inhibitor.

Combination with CYP3A4 Inhibitors

Disclosed herein, in certain embodiments, are pharmaceutical combinations comprising a Btk inhibitor compound and a CYP3A4 inhibitor.

Further disclosed herein, in certain embodiments, are pharmaceutical combinations comprising Ibrutinib and a CYP3A4 inhibitor.

Cytochrome P450 3A4 (abbreviated CYP3A4) (EC 1.14.13.97), is a member of the cytochrome P450 mixed-function oxidase system. Cytochrome P450 proteins are monooxygenases that catalyze many reactions involved in drug metabolism. CYP3A4 is encoded by the CYP3A4 gene. This gene is part of a cluster of cytochrome P450 genes on chromosome 7q21.1. CYP3A4 is involved in the oxidation of a large range of substrates, for example Ibrutinib.

Cytochrome P450 enzymes modify a variety of substrate, including hydroxylation, epoxidation, aromatic oxidations, heteroatom oxidations, N- and O-dealkylations, aldehyde oxidations, and dehydrogenations.

In some embodiments, Ibrutinib and a CYP3A4 inhibitor are co-administration concurrently (e.g., simultaneously, essentially simultaneously or within the same treatment protocol) or sequentially.

In some embodiments, Ibrutinib and a CYP3A4 inhibitor are co-administered in separate dosage forms. In some embodiments, Ibrutinib and a CYP3A4 inhibitor are co-administered in combined dosage forms.

In some embodiments, the co-administration of Ibrutinib and a CYP3A4 inhibitor increases the oral bioavailability of Ibrutinib. In some embodiments, the co-administration of Ibrutinib and a CYP3A4 inhibitor increases the Cmax of Ibrutinib. In some embodiments, the co-administration of Ibrutinib and a CYP3A4 inhibitor increases the AUC of Ibrutinib.

In some embodiments, co-administration of Ibrutinib and a CYP3A4 inhibitor increases the Cmax of Ibrutinib by about 20× to about 40× the Cmax of Ibrutinib administered without a CYP3A4 inhibitor. In some embodiments, co-administration of Ibrutinib and a CYP3A4 inhibitor increases the Cmax of Ibrutinib by about 25× to about 35×. In some embodiments, co-administration of Ibrutinib and a CYP3A4 inhibitor increases the Cmax of Ibrutinib by about 20×. In some embodiments, co-administration of Ibrutinib and a CYP3A4 inhibitor increases the Cmax of Ibrutinib by about 21×. In some embodiments, co-administration of Ibrutinib and a CYP3A4 inhibitor increases the Cmax of Ibrutinib by about 22×. In some embodiments, co-administration of Ibrutinib and a CYP3A4 inhibitor increases the Cmax of Ibrutinib by about 23×. In some embodiments, co-administration of Ibrutinib and a CYP3A4 inhibitor increases the Cmax of Ibrutinib by about 24×. In some embodiments, co-administration of Ibrutinib and a CYP3A4 inhibitor increases the Cmax of Ibrutinib by about 25×. In some embodiments, co-administration of Ibrutinib and a CYP3A4 inhibitor increases the Cmax of Ibrutinib by about 26×. In some embodiments, co-administration of Ibrutinib and a CYP3A4 inhibitor increases the Cmax of Ibrutinib by about 27×. In some embodiments, co-administration of Ibrutinib and a CYP3A4 inhibitor increases the Cmax of Ibrutinib by about 28×. In some embodiments, co-administration of Ibrutinib and a CYP3A4 inhibitor increases the Cmax of Ibrutinib by about 29×. In some embodiments, co-administration of Ibrutinib and a CYP3A4 inhibitor increases the Cmax of Ibrutinib by about 30×. In some embodiments, co-administration of Ibrutinib and a CYP3A4 inhibitor increases the Cmax of Ibrutinib by about 31×. In some embodiments, co-administration of Ibrutinib and a CYP3A4 inhibitor increases the Cmax of Ibrutinib by about 32×. In some embodiments, co-administration of Ibrutinib and a CYP3A4 inhibitor increases the Cmax of Ibrutinib by about 33×. In some embodiments, co-administration of Ibrutinib and a CYP3A4 inhibitor increases the Cmax of Ibrutinib by about 34×. In some embodiments, co-administration of Ibrutinib and a CYP3A4 inhibitor increases the Cmax of Ibrutinib by about 35×. In some embodiments, co-administration of Ibrutinib and a CYP3A4 inhibitor increases the Cmax of Ibrutinib by about 36×. In some embodiments, co-administration of Ibrutinib and a CYP3A4 inhibitor increases the Cmax of Ibrutinib by about 37×. In some embodiments, co-administration of Ibrutinib and a CYP3A4 inhibitor increases the Cmax of Ibrutinib by about 38×. In some embodiments, co-administration of Ibrutinib and a CYP3A4 inhibitor increases the Cmax of Ibrutinib by about 39×. In some embodiments, co-administration of Ibrutinib and a CYP3A4 inhibitor increases the Cmax of Ibrutinib by about 40×.

In some embodiments, the co-administration of Ibrutinib and a CYP3A4 inhibitor increases the AUC of Ibrutinib by about 15× to about 35× the AUC of Ibrutinib administered without a CYP3A4 inhibitor. In some embodiments, co-administration of Ibrutinib and a CYP3A4 inhibitor increases the AUC of Ibrutinib by about 20× to about 30×. In some embodiments, co-administration of Ibrutinib and a CYP3A4 inhibitor increases the AUC of Ibrutinib by about 2× to about 35× the AUC of Ibrutinib administered without a CYP3A4 inhibitor. In some embodiments, co-administration of Ibrutinib and a CYP3A4 inhibitor increases the AUC of Ibrutinib by about 2× to about 30× the AUC of Ibrutinib administered without a CYP3A4 inhibitor. In some embodiments, co-administration of Ibrutinib and a CYP3A4 inhibitor increases the AUC of Ibrutinib by about 2× to about 25× the AUC of Ibrutinib administered without a CYP3A4 inhibitor. In some embodiments, co-administration of Ibrutinib and a CYP3A4 inhibitor increases the AUC of Ibrutinib by about 2× to about 20× the AUC of Ibrutinib administered without a CYP3A4 inhibitor. In some embodiments, co-administration of Ibrutinib and a CYP3A4 inhibitor increases the AUC of Ibrutinib by about 2× to about 15× the AUC of Ibrutinib administered without a CYP3A4 inhibitor. In some embodiments, co-administration of Ibrutinib and a CYP3A4 inhibitor increases the AUC of Ibrutinib by about 2× to about 10× the AUC of Ibrutinib administered without a CYP3A4 inhibitor. In some embodiments, co-administration of Ibrutinib and a CYP3A4 inhibitor increases the AUC of Ibrutinib by about 2× to about 5× the AUC of Ibrutinib administered without a CYP3A4 inhibitor. In some embodiments, co-administration of Ibrutinib and a CYP3A4 inhibitor increases the AUC of Ibrutinib by about 2× to about 4× the AUC of Ibrutinib administered without a CYP3A4 inhibitor. In some embodiments, co-administration of Ibrutinib and a CYP3A4 inhibitor increases the AUC of Ibrutinib by about 15×. In some embodiments, co-administration of Ibrutinib and a CYP3A4 inhibitor increases the AUC of Ibrutinib by about 2×. In some embodiments, co-administration of Ibrutinib and a CYP3A4 inhibitor increases the AUC of Ibrutinib by about 3×. In some embodiments, co-administration of Ibrutinib and a CYP3A4 inhibitor increases the AUC of Ibrutinib by about 4×. In some embodiments, co-administration of Ibrutinib and a CYP3A4 inhibitor increases the AUC of Ibrutinib by about 5×. In some embodiments, co-administration of Ibrutinib and a CYP3A4 inhibitor increases the AUC of Ibrutinib by about 6×. In some embodiments, co-administration of Ibrutinib and a CYP3A4 inhibitor increases the AUC of Ibrutinib by about 7×. In some embodiments, co-administration of Ibrutinib and a CYP3A4 inhibitor increases the AUC of Ibrutinib by about 8×. In some embodiments, co-administration of Ibrutinib and a CYP3A4 inhibitor increases the AUC of Ibrutinib by about 9×. In some embodiments, co-administration of Ibrutinib and a CYP3A4 inhibitor increases the AUC of Ibrutinib by about 10×. In some embodiments, co-administration of Ibrutinib and a CYP3A4 inhibitor increases the AUC of Ibrutinib by about 11×. In some embodiments, co-administration of Ibrutinib and a CYP3A4 inhibitor increases the AUC of Ibrutinib by about 12×. In some embodiments, co-administration of Ibrutinib and a CYP3A4 inhibitor increases the AUC of Ibrutinib by about 13×. In some embodiments, co-administration of Ibrutinib and a CYP3A4 inhibitor increases the AUC of Ibrutinib by about 14×. In some embodiments, co-administration of Ibrutinib and a CYP3A4 inhibitor increases the AUC of Ibrutinib by about 15×. In some embodiments, co-administration of Ibrutinib and a CYP3A4 inhibitor increases the AUC of Ibrutinib by about 16×. In some embodiments, co-administration of Ibrutinib and a CYP3A4 inhibitor increases the AUC of Ibrutinib by about 17×. In some embodiments, co-administration of Ibrutinib and a CYP3A4 inhibitor increases the AUC of Ibrutinib by about 18×. In some embodiments, co-administration of Ibrutinib and a CYP3A4 inhibitor increases the AUC of Ibrutinib by about 19×. In some embodiments, co-administration of Ibrutinib and a CYP3A4 inhibitor increases the AUC of Ibrutinib by about 20×. In some embodiments, co-administration of Ibrutinib and a CYP3A4 inhibitor increases the AUC of Ibrutinib by about 21×. In some embodiments, co-administration of Ibrutinib and a CYP3A4 inhibitor increases the AUC of Ibrutinib by about 22×. In some embodiments, co-administration of Ibrutinib and a CYP3A4 inhibitor increases the AUC of Ibrutinib by about 23×. In some embodiments, co-administration of Ibrutinib and a CYP3A4 inhibitor increases the AUC of Ibrutinib by about 24×. In some embodiments, co-administration of Ibrutinib and a CYP3A4 inhibitor increases the AUC of Ibrutinib by about 25×. In some embodiments, co-administration of Ibrutinib and a CYP3A4 inhibitor increases the AUC of Ibrutinib by about 26×. In some embodiments, co-administration of Ibrutinib and a CYP3A4 inhibitor increases the AUC of Ibrutinib by about 27×. In some embodiments, co-administration of Ibrutinib and a CYP3A4 inhibitor increases the AUC of Ibrutinib by about 28×. In some embodiments, co-administration of Ibrutinib and a CYP3A4 inhibitor increases the AUC of Ibrutinib by about 29×. In some embodiments, co-administration of Ibrutinib and a CYP3A4 inhibitor increases the AUC of Ibrutinib by about 30×. In some embodiments, co-administration of Ibrutinib and a CYP3A4 inhibitor increases the AUC of Ibrutinib by about 31×. In some embodiments, co-administration of Ibrutinib and a CYP3A4 inhibitor increases the AUC of Ibrutinib by about 32×. In some embodiments, co-administration of Ibrutinib and a CYP3A4 inhibitor increases the AUC of Ibrutinib by about 33×. In some embodiments, co-administration of Ibrutinib and a CYP3A4 inhibitor increases the AUC of Ibrutinib by about 34×. In some embodiments, co-administration of Ibrutinib and a CYP3A4 inhibitor increases the AUC of Ibrutinib by about 35×.

In some embodiments, co-administration of Ibrutinib and a CYP3A4 inhibitor does not significantly affect the Tmax or T1/2 of Ibrutinib as compared to the Tmax and T1/2 of Ibrutinib administered without a CYP3A4 inhibitor.

In some embodiments, the daily dosage of Ibrutinib when administered in combination with a CYP3A4 inhibitor is about 10 mg to about 100 mg. In some embodiments, the daily dosage of Ibrutinib when administered in combination with a CYP3A4 inhibitor is about 10, mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the daily dosage of Ibrutinib when administered in combination with a CYP3A4 inhibitor is about 40 mg to about 70 mg. In some embodiments, the daily dosage of Ibrutinib when administered in combination with a CYP3A4 inhibitor is about 40 mg.

Any suitable daily dose of a CYP3A4 inhibitor is contemplated for use with the compositions, dosage forms, and methods disclosed herein. Daily dose of the CYP3A4 inhibitor depends on multiple factors, the determination of which is within the skills of one of skill in the art. For example, the daily dose of the CYP3A4 inhibitor depends of the strength of the CYP3A4 inhibitor. Weak CYP3A4 inhibitors (e.g. cimetidine) will require higher daily doses than moderate CYP3A4 inhibitors (e.g., erythromycin, grapefruit juice, verapamil, diltiazem), and moderate CYP3A4 inhibitors will require higher daily doses than strong CYP3A4 inhibitors (e.g., indinavir, nelfinavir, ritonavir, clarithromycin, itraconazole, ketoconazole, nefazodone).

### Exemplary CYP3A4 Inhibitors

In some embodiments, Ibrutinib is co-administered with an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof.

In some embodiments, Ibrutinib is co-administered with alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; troleandromycin, vincristine; voriconazole; or any combinations thereof. In some embodiments, Ibrutinib is co-administered with cobicistat (GS-9350) or analogs or derivatives of cobicistat (GS-9350). In some embodiments, Ibrutinib is co-administered with ketoconazole. In some embodiments, Ibrutinib is co-administered with ritonavir.

Diazepam→3-OH refers to 3-hydroxydiazepam and quinidine→3-OH refers to 3-hydroxyquinidine.

Any suitable CYP3A4 inhibitor is contemplated for use with the compositions, dosage forms, and methods disclosed herein. The selection of the CYP3A4 inhibitor depends on multiple factors, and the selection of the CYP3A4 inhibitor is within the skills of one of skill in the art. For example, factors to be considered include the desired reduction in the daily dose of Ibrutinib, any additional drug interactions of the CYP3A4 inhibitor, and the length for which the CYP3A4 inhibitor may be taken. In certain instances, the CYP3A4 inhibitor is a CYP3A4 inhibitor which may be taken long-term, for example chronically.

Disclosed herein, in certain embodiments, are methods of increasing the Cmax of ibruitinib comprising co-administering a combination of Ibrutinib and a CYP3A4 inhibitor. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor.

Disclosed herein, in certain embodiments, are methods of increasing the AUC of Ibrutinib comprising administering a combination of Ibrutinib and a CYP3A4 inhibitor. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method does not significantly affect the Tmax or T1/2 of Ibrutinib as compared to the Tmax and T1/2 of Ibrutinib administered without a CYP3A4 inhibitor.

### Methods of Use

### B-Cell Proliferative Disorders

In some embodiments is a method of treating a cancer in an individual in need thereof comprising administering a combination of a Btk inhibitor and a CYP3A4 inhibitor.

In some embodiments is a method of treating a cancer in an individual in need thereof comprising administering a combination of Ibrutinib and a CYP3A4 inhibitor. In some embodiments, the cancer is a B-cell proliferative disorder. In some embodiments, the cancer is chronic lymphocytic leukemia (CLL), small lymphocytic lymphoma (SLL), high risk CLL, or a non-CLL/SLL lymphoma. In some embodiments, the cancer is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the cancer is acute or chronic myelogenous (or myeloid) leukemia, myelodysplastic syndrome, or acute lymphoblastic leukemia. In some embodiments, the cancer is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the cancer is high risk CLL or high risk SLL. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor. In some embodiments, Ibrutinib and the CYP3A4 inhibitor are in a combined dosage form. In some embodiments, Ibrutinib and the CYP3A4 inhibitor are in separate dosage forms. In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially. In some embodiments, the methods further comprise co-administering chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the methods further comprise co-administering cyclophosphamide, hydroxydaunorubicin, vincristine, and prednisone, and optionally, rituximab. In some embodiments, the methods further comprise co-administering bendamustine, and rituximab. In some embodiments, the methods further comprise co-administering fludarabine, cyclophosphamide, and rituximab. In some embodiments, the methods further comprise co-administering cyclophosphamide, vincristine, and prednisone, and optionally, rituximab. In some embodiments, the methods further comprise co-administering etoposide, doxorubicin, vincristine, cyclophosphamide, prednisolone, and optionally, rituximab. In some embodiments, the methods further comprise co-administering dexamethasone and lenalidomide. In some embodiments, Ibrutinib is amorphous or crystalline.

B-cell proliferative disorders (BCPDs) are neoplasms of the blood and encompass, inter alia, non-Hodgkin lymphoma, multiple myeloma, and leukemia. BCPDs can originate either in the lymphatic tissues (as in the case of lymphoma) or in the bone marrow (as in the case of leukemia and myeloma), and they all are involved with the uncontrolled growth of lymphocytes or white blood cells. There are many subtypes of BCPD, e.g., chronic lymphocytic leukemia (CLL) and non-Hodgkin lymphoma (NHL). The disease course and treatment of BCPD is dependent on the BCPD subtype; however, even within each subtype the clinical presentation, morphologic appearance, and response to therapy is heterogeneous.

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment (Freedman et al., “Non-Hodgkin's Lymphomas” Chapter 134, Cancer Medicine, (an approved publication of the American Cancer Society, B.C. Decker Inc., Hamilton, Ontario, 2003).

### Non-Hodgkin's Lymphomas

Disclosed herein, in certain embodiments, is a method for treating a non-Hodgkin's lymphoma in an individual in need thereof, comprising: administering a combination of a Btk inhibitor and a CYP3A4 inhibitor.

Disclosed herein, in certain embodiments, is a method for treating a non-Hodgkin's lymphoma in an individual in need thereof, comprising: administering a combination of Ibrutinib and a CYP3A4 inhibitor.

Further disclosed herein, in certain embodiments, is a method for treating relapsed or refractory non-Hodgkin's lymphoma in an individual in need thereof, comprising: administering to the individual a combination of a Btk inhibitor and a CYP3A4 inhibitor. In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

Further disclosed herein, in certain embodiments, is a method for treating relapsed or refractory non-Hodgkin's lymphoma in an individual in need thereof, comprising: administering to the individual a combination of Ibrutinib and a CYP3A4 inhibitor. In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

Non-Hodgkin lymphomas (NHL) are a diverse group of malignancies that are predominately of B-cell origin. NHL may develop in any organs associated with lymphatic system such as spleen, lymph nodes or tonsils and can occur at any age. NHL is often marked by enlarged lymph nodes, fever, and weight loss. NHL is classified as either B-cell or T-cell NHL. Lymphomas related to lymphoproliferative disorders following bone marrow or stem cell transplantation are usually B-cell NHL. In the Working Formulation classification scheme, NHL has been divided into low-, intermediate-, and high-grade categories by virtue of their natural histories (see “The Non-Hodgkin's Lymphoma Pathologic Classification Project,” Cancer 49(1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. Additional non-Hodgkin's lymphomas are contemplated within the scope of the present invention and apparent to those of ordinary skill in the art. DLBCL

Disclosed herein, in certain embodiments, is a method for treating a DLCBL in an individual in need thereof, comprising: administering a combination of a Btk inhibitor and a CYP3A4 inhibitor.

Further disclosed herein, in certain embodiments, is a method for treating a DLCBL in an individual in need thereof, comprising: administering a combination of Ibrutinib and a CYP3A4 inhibitor.

As used herein, the term “Diffuse large B-cell lymphoma (DLBCL)” refers to a neoplasm of the germinal center B lymphocytes with a diffuse growth pattern and a high-intermediate proliferation index. DLBCLs represent approximately 30% of all lymphomas and may present with several morphological variants including the centroblastic, immunoblastic, T-cell/histiocyte rich, anaplastic and plasmoblastic subtypes. Genetic tests have shown that there are different subtypes of DLBCL. These subtypes seem to have different outlooks (prognoses) and responses to treatment. DLBCL can affect any age group but occurs mostly in older people (the average age is mid-60s).

Disclosed herein, in certain embodiments, is a method for treating diffuse large B-cell lymphoma, activated B cell-like subtype (ABC-DLBCL), in an individual in need thereof, comprising: administering to the individual a combination of Ibrutinib and a CYP3A4 inhibitor. The ABC subtype of diffuse large B-cell lymphoma (ABC-DLBCL) is thought to arise from post germinal center B cells that are arrested during plasmatic differentiation. The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation.

A particularly relevant signaling pathway in the pathogenesis of ABC-DLBCL is the one mediated by the nuclear factor (NF)-κB transcription complex. The NF-κB family comprises 5 members (p50, p52, p65, c-rel and Re1B) that form homo- and heterodimers and function as transcriptional factors to mediate a variety of proliferation, apoptosis, inflammatory and immune responses and are critical for normal B-cell development and survival. NF-κB is widely used by eukaryotic cells as a regulator of genes that control cell proliferation and cell survival. As such, many different types of human tumors have misregulated NF-κB: that is, NF-κB is constitutively active. Active NF-κB turns on the expression of genes that keep the cell proliferating and protect the cell from conditions that would otherwise cause it to die via apoptosis.

The dependence of ABC DLBCLs on NF-kB depends on a signaling pathway upstream of IkB kinase comprised of CARD11, BCL10 and MALT1 (the CBM complex). Interference with the CBM pathway extinguishes NF-kB signaling in ABC DLBCL cells and induces apoptosis. The molecular basis for constitutive activity of the NF-kB pathway is a subject of current investigation but some somatic alterations to the genome of ABC DLBCLs clearly invoke this pathway. For example, somatic mutations of the coiled-coil domain of CARD11 in DLBCL render this signaling scaffold protein able to spontaneously nucleate protein-protein interaction with MALT1 and BCL10, causing IKK activity and NF-kB activation. Constitutive activity of the B cell receptor signaling pathway has been implicated in the activation of NF-kB in ABC DLBCLs with wild type CARD11, and this is associated with mutations within the cytoplasmic tails of the B cell receptor subunits CD79A and CD79B. Oncogenic activating mutations in the signaling adapter MYD88 activate NF-kB and synergize with B cell receptor signaling in sustaining the survival of ABC DLBCL cells. In addition, inactivating mutations in a negative regulator of the NF-kB pathway, A20, occur almost exclusively in ABC DLBCL.

Indeed, genetic alterations affecting multiple components of the NF-κB signaling pathway have been recently identified in more than 50% of ABC-DLBCL patients, where these lesions promote constitutive NF-κB activation, thereby contributing to lymphoma growth. These include mutations of CARD11 (˜10% of the cases), a lymphocyte-specific cytoplasmic scaffolding protein that—together with MALT1 and BCL10—forms the BCR signalosome, which relays signals from antigen receptors to the downstream mediators of NF-κB activation. An even larger fraction of cases (˜30%) carry biallelic genetic lesions inactivating the negative NF-κregulator A20. Further, high levels of expression of NF-κB target genes have been observed in ABC-DLBCL tumor samples. See, e.g., U. Klein et al., (2008), Nature Reviews Immunology 8:22-23; R. E. Davis et al., (2001), Journal of Experimental Medicine 194:1861-1874; G. Lentz et al., (2008), Science 319:1676-1679; M. Compagno et al., (2009), Nature 459:712-721; and L. Srinivasan et al., (2009), Cell 139:573-586).

DLBCL cells of the ABC subtype, such as OCI-Ly10, have chronic active BCR signaling and are very sensitive to the Btk inhibitor described herein. The irreversible Btk inhibitor described herein potently and irreversibly inhibits the growth of OCI-Ly10 (EC50 continuous exposure=10 nM, EC50 1 hour pulse=50 nM). In addition, induction of apoptosis, as shown by capsase activation, Annexin-V flow cytometry and increase in sub-GO fraction is observed in OCILy10. Both sensitive and resistant cells express Btk at similar levels, and the active site of Btk is fully occupied by the inhibitor in both as shown using a fluorescently labeled affinity probe. OCI-Ly10 cells are shown to have chronically active BCR signaling to NF-kB which is dose dependently inhibited by the Btk inhibitors described herein. The activity of Btk inhibitors in the cell lines studied herein are also characterized by comparing signal transduction profiles (Btk, PLCy, ERK, NF-kB, AKT), cytokine secretion profiles and mRNA expression profiles, both with and without BCR stimulation, and observed significant differences in these profiles that lead to clinical biomarkers that identify the most sensitive patient populations to Btk inhibitor treatment. See U.S. Pat. No. 7,711,492 and Staudt et al., Nature, Vol. 463, Jan. 7, 2010, pp. 88-92, the contents of which are incorporated by reference in their entirety.

### Follicular Lymphoma

Disclosed herein, in certain embodiments, is a method for treating a follicular lymphoma in an individual in need thereof, comprising: administering a combination of a Btk inhibitor and a CYP3A4 inhibitor.

Further disclosed herein, in certain embodiments, is a method for treating a follicular lymphoma in an individual in need thereof, comprising: administering a combination of Ibrutinib and a CYP3A4 inhibitor.

As used herein, the term “follicular lymphoma” refers to any of several types of non-Hodgkin's lymphoma in which the lymphomatous cells are clustered into nodules or follicles. The term follicular is used because the cells tend to grow in a circular, or nodular, pattern in lymph nodes. The average age for people with this lymphoma is about 60.

### CLL/SLL

Disclosed herein, in certain embodiments, is a method for treating a CLL or SLL in an individual in need thereof, comprising: administering a combination of a Btk inhibitor and a CYP3A4 inhibitor.

Further disclosed herein, in certain embodiments, is a method for treating a CLL or SLL in an individual in need thereof, comprising: administering a combination of Ibrutinib and a CYP3A4 inhibitor.

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL. SLL accounts for about 5% to 10% of all lymphomas. When most of the cancer cells are in the bloodstream and the bone marrow, it is called CLL.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. It is estimated that 100,760 people in the United States are living with or are in remission from CLL. Most (>75%) people newly diagnosed with CLL are over the age of 50. Currently CLL treatment focuses on controlling the disease and its symptoms rather than on an outright cure. CLL is treated by chemotherapy, radiation therapy, biological therapy, or bone marrow transplantation. Symptoms are sometimes treated surgically (splenectomy removal of enlarged spleen) or by radiation therapy (“de-bulking” swollen lymph nodes). Though CLL progresses slowly in most cases, it is considered generally incurable. Certain CLLs are classified as high-risk. As used herein, “high risk CLL” means CLL characterized by at least one of the following 1) 17p13-; 2) 11q22-; 3) unmutated IgVH together with ZAP-70+and/or CD38+; or 4) trisomy 12.

CLL treatment is typically administered when the patient's clinical symptoms or blood counts indicate that the disease has progressed to a point where it may affect the patient's quality of life.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

### Mantle Cell Lymphoma

Disclosed herein, in certain embodiments, is a method for treating a Mantle cell lymphoma in an individual in need thereof, comprising: administering a combination of a Btk inhibitor and a CYP3A4 inhibitor.

Further disclosed herein, in certain embodiments, is a method for treating a Mantle cell lymphoma in an individual in need thereof, comprising: administering a combination of Ibrutinib and a CYP3A4 inhibitor.

As used herein, the term, “Mantle cell lymphoma” refers to a subtype of B-cell lymphoma, due to CD5 positive antigen-naive pregerminal center B-cell within the mantle zone that surrounds normal germinal center follicles. MCL cells generally over-express cyclin D1 due to a t(11:14) chromosomal translocation in the DNA. More specifically, the translocation is at t(11;14)(q13;q32). Only about 5% of lymphomas are of this type. The cells are small to medium in size. Men are affected most often. The average age of patients is in the early 60s. The lymphoma is usually widespread when it is diagnosed, involving lymph nodes, bone marrow, and, very often, the spleen. Mantle cell lymphoma is not a very fast growing lymphoma, but is difficult to treat.

### Marginal Zone B-cell Lymphoma

Disclosed herein, in certain embodiments, is a method for treating a marginal zone B-cell lymphoma in an individual in need thereof, comprising: administering a combination of a Btk inhibitor and a CYP3A4 inhibitor.

Further disclosed herein, in certain embodiments, is a method for treating a marginal zone B-cell lymphoma in an individual in need thereof, comprising: administering a combination of Ibrutinib and a CYP3A4 inhibitor.

As used herein, the term “marginal zone B-cell lymphoma” refers to a group of related B-cell neoplasms that involve the lymphoid tissues in the marginal zone, the patchy area outside the follicular mantle zone. Marginal zone lymphomas account for about 5% to 10% of lymphomas. The cells in these lymphomas look small under the microscope. There are 3 main types of marginal zone lymphomas including extranodal marginal zone B-cell lymphomas, nodal marginal zone B-cell lymphoma, and splenic marginal zone lymphoma.

### MALT

Disclosed herein, in certain embodiments, is a method for treating a MALT in an individual in need thereof, comprising: administering a combination of a Btk inhibitor and a CYP3A4 inhibitor.

Further disclosed herein, in certain embodiments, is a method for treating a MALT in an individual in need thereof, comprising: administering a combination of Ibrutinib and a CYP3A4 inhibitor.

The term “mucosa-associated lymphoid tissue (MALT) lymphoma”, as used herein, refers to extranodal manifestations of marginal-zone lymphomas. Most MALT lymphoma are a low grade, although a minority either manifest initially as intermediate-grade non-Hodgkin lymphoma (NHL) or evolve from the low-grade form. Most of the MALT lymphoma occur in the stomach, and roughly 70% of gastric MALT lymphoma are associated with Helicobacter pylori infection. Several cytogenetic abnormalities have been identified, the most common being trisomy 3 or t(11;18). Many of these other MALT lymphoma have also been linked to infections with bacteria or viruses. The average age of patients with MALT lymphoma is about 60.

### Nodal Marginal Zone B-Cell Lymphoma

Disclosed herein, in certain embodiments, is a method for treating a nodal marginal zone B-cell lymphoma in an individual in need thereof, comprising: administering a combination of a Btk inhibitor and a CYP3A4 inhibitor.

Further disclosed herein, in certain embodiments, is a method for treating a nodal marginal zone B-cell lymphoma in an individual in need thereof, comprising: administering a combination of Ibrutinib and a CYP3A4 inhibitor.

The term “nodal marginal zone B-cell lymphoma” refers to an indolent B-cell lymphoma that is found mostly in the lymph nodes. The disease is rare and only accounts for 1% of all Non-Hodgkin's Lymphomas (NHL). It is most commonly diagnosed in older patients, with women more susceptible than men. The disease is classified as a marginal zone lymphoma because the mutation occurs in the marginal zone of the B-cells. Due to its confinement in the lymph nodes, this disease is also classified as nodal.

### Splenic Marginal Zone B-Cell Lymphoma

Disclosed herein, in certain embodiments, is a method for treating a splenic marginal zone B-cell lymphoma in an individual in need thereof, comprising: administering a combination of a Btk inhibitor and a CYP3A4 inhibitor.

Further disclosed herein, in certain embodiments, is a method for treating a splenic marginal zone B-cell lymphoma in an individual in need thereof, comprising: administering a combination of Ibrutinib and a CYP3A4 inhibitor.

The term “splenic marginal zone B-cell lymphoma” refers to specific low-grade small B-cell lymphoma that is incorporated in the World Health Organization classification. Characteristic features are splenomegaly, moderate lymphocytosis with villous morphology, intrasinusoidal pattern of involvement of various organs, especially bone marrow, and relative indolent course. Tumor progression with increase of blastic forms and aggressive behavior are observed in a minority of patients. Molecular and cytogenetic studies have shown heterogeneous results probably because of the lack of standardized diagnostic criteria.

### Burkitt Lymphoma

Disclosed herein, in certain embodiments, is a method for treating a Burkitt lymphoma in an individual in need thereof, comprising: administering a combination of a Btk inhibitor and a CYP3A4 inhibitor.

Further disclosed herein, in certain embodiments, is a method for treating a Burkitt lymphoma in an individual in need thereof, comprising: administering a combination of Ibrutinib and a CYP3A4 inhibitor.

The term “Burkitt lymphoma” refers to a type of Non-Hodgkin Lymphoma (NHL) that commonly affects children. It is a highly aggressive type of B-cell lymphoma that often starts and involves body parts other than lymph nodes. In spite of its fast-growing nature, Burkitt's lymphoma is often curable with modern intensive therapies. There are two broad types of Burkitt's lymphoma—the sporadic and the endemic varieties:

Endemic Burkitt's lymphoma: The disease involves children much more than adults, and is related to Epstein Barr Virus (EBV) infection in 95% cases. It occurs primarily is equatorial Africa, where about half of all childhood cancers are Burkitt's lymphoma. It characteristically has a high chance of involving the jawbone, a rather distinctive feature that is rare in sporadic Burkitt's. It also commonly involves the abdomen.

Sporadic Burkitt's lymphoma: The type of Burkitt's lymphoma that affects the rest of the world, including Europe and the Americas is the sporadic type. Here too, it's mainly a disease in children. The link between Epstein Barr Virus (EBV) is not as strong as with the endemic variety, though direct evidence of EBV infection is present in one out of five patients. More than the involvement of lymph nodes, it is the abdomen that is notably affected in more than 90% of the children. Bone marrow involvement is more common than in the sporadic variety.

### Waldenstrom Macroglobulinemia

Disclosed herein, in certain embodiments, is a method for treating a Waldenstrom macroglobulinemia in an individual in need thereof, comprising: administering a combination of a Btk inhibitor and a CYP3A4 inhibitor.

Further disclosed herein, in certain embodiments, is a method for treating a Waldenstrom macroglobulinemia in an individual in need thereof, comprising: administering a combination of Ibrutinib and a CYP3A4 inhibitor.

The term “Waldenstrom macroglobulinemia”, also known as lymphoplasmacytic lymphoma, is cancer involving a subtype of white blood cells called lymphocytes. It is characterized by an uncontrolled clonal proliferation of terminally differentiated B lymphocytes. It is also characterized by the lymphoma cells making an antibody called immunoglobulin M (IgM). The IgM antibodies circulate in the blood in large amounts, and cause the liquid part of the blood to thicken, like syrup. This can lead to decreased blood flow to many organs, which can cause problems with vision (because of poor circulation in blood vessels in the back of the eyes) and neurological problems (such as headache, dizziness, and confusion) caused by poor blood flow within the brain. Other symptoms can include feeling tired and weak, and a tendency to bleed easily. The underlying etiology is not fully understood but a number of risk factors have been identified, including the locus 6p21.3 on chromosome 6. There is a 2- to 3-fold risk increase of developing WM in people with a personal history of autoimmune diseases with autoantibodies and particularly elevated risks associated with hepatitis, human immunodeficiency virus, and rickettsiosis.

### Multiple Myeloma

Disclosed herein, in certain embodiments, is a method for treating a myeloma in an individual in need thereof, comprising: administering a combination of a Btk inhibitor and a CYP3A4 inhibitor.

Further disclosed herein, in certain embodiments, is a method for treating a myeloma in an individual in need thereof, comprising: administering a combination of Ibrutinib and a CYP3A4 inhibitor.

Multiple myeloma, also known as MM, myeloma, plasma cell myeloma, or as Kahler's disease (after Otto Kahler) is a cancer of the white blood cells known as plasma cells. A type of B cell, plasma cells are a crucial part of the immune system responsible for the production of antibodies in humans and other vertebrates. They are produced in the bone marrow and are transported through the lymphatic system.

### Leukemia

Disclosed herein, in certain embodiments, is a method for treating a leukemia in an individual in need thereof, comprising: administering a combination of a Btk inhibitor and a CYP3A4 inhibitor.

Further disclosed herein, in certain embodiments, is a method for treating a leukemia in an individual in need thereof, comprising: administering a combination of Ibrutinib and a CYP3A4 inhibitor.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, Acute lymphoblastic leukemia (ALL), Acute myelogenous leukemia (AML), Chronic myelogenous leukemia (CML), and Hairy cell leukemia (HCL).

Symptoms, diagnostic tests, and prognostic tests for each of the above-mentioned conditions are known. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models are useful for establishing a range of therapeutically effective doses of irreversible Btk inhibitor compounds, such as Ibrutinib, for treating any of the foregoing diseases.

The therapeutic efficacy of Ibrutinib for any one of the foregoing diseases can be optimized during a course of treatment. For example, a subject being treated can undergo a diagnostic evaluation to correlate the relief of disease symptoms or pathologies to inhibition of in vivo Btk activity achieved by administering a given dose of Ibrutinib. Cellular assays known in the art can be used to determine in vivo activity of Btk in the presence or absence of an irreversible Btk inhibitor. For example, since activated Btk is phosphorylated at tyrosine 223 (Y223) and tyrosine 551 (Y551), phospho-specific immunocytochemical staining of P-Y223 or P-Y551-positive cells can be used to detect or quantify activation of Btk in a population of cells (e.g., by FACS analysis of stained vs unstained cells). See, e.g., Nisitani et al. (1999), Proc. Natl. Acad. Sci, USA 96:2221-2226. Thus, the amount of the Btk inhibitor compound that is administered to a subject can be increased or decreased as needed so as to maintain a level of Btk inhibition optimal for treating the subject's disease state.

Ibrutinib can irreversibly inhibit Btk and may be used to treat mammals suffering from Bruton's tyrosine kinase-dependent or Bruton's tyrosine kinase mediated conditions or diseases, including, but not limited to, cancer, autoimmune and other inflammatory diseases. Ibrutinib has shown efficacy is a wide variety of diseases and conditions that are described herein.

In some embodiments, a Btk inhibitor and a CYP3A4 inhibitor are used for the manufacture of a medicament for treating any of the foregoing conditions (e.g., autoimmune diseases, inflammatory diseases, allergy disorders, B-cell proliferative disorders, or thromboembolic disorders).

In some embodiments, Ibrutinib and a CYP3A4 inhibitor are used for the manufacture of a medicament for treating any of the foregoing conditions (e.g., autoimmune diseases, inflammatory diseases, allergy disorders, B-cell proliferative disorders, or thromboembolic disorders).

### Further Uses

Disclosed herein, in certain embodiments, are methods method of treating an autoimmune disorder in an individual in need thereof comprising administering a combination of a Btk inhibitor and a CYP3A4 inhibitor. Further disclosed herein, in certain embodiments, are methods method of treating an autoimmune disorder in an individual in need thereof comprising administering a combination of Ibrutinib and a CYP3A4 inhibitor. In some embodiments, the autoimmune disorder is rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease, Sjogren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, vulvodynia, or any combinations thereof. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; any analogs thereof; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor. In some embodiments, Ibrutinib and the CYP3A4 inhibitor are in a combined dosage form. In some embodiments, Ibrutinib and the CYP3A4 inhibitor are in separate dosage forms. In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially. In some embodiments, Ibrutinib is amorphous or crystalline.

Disclosed herein, in certain embodiments, are methods of treating a heteroimmune disorder in an individual in need thereof comprising administering a combination of a Btk inhibitor and a CYP3A4 inhibitor. Further disclosed herein, in certain embodiments, are methods of treating a heteroimmune disorder in an individual in need thereof comprising administering a combination of Ibrutinib and a CYP3A4 inhibitor. In some embodiments, the heteroimmune disorder is graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, atopic dermatitis, or any combinations thereof. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor. In some embodiments, Ibrutinib and the CYP3A4 inhibitor are in a combined dosage form. In some embodiments, Ibrutinib and the CYP3A4 inhibitor are in separate dosage forms. In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially. In some embodiments, the methods further comprise co-administering dexamethasone and lenalidomide. In some embodiments, Ibrutinib is amorphous or crystalline.

Disclosed herein, in certain embodiments, are methods of treating an inflammatory disorder in an individual in need thereof comprising administering a combination of a Btk inhibitor and a CYP3A4 inhibitor. Further disclosed herein, in certain embodiments, are methods of treating an inflammatory disorder in an individual in need thereof comprising administering a combination of Ibrutinib and a CYP3A4 inhibitor. In some embodiments, the inflammatory disorder is asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, vulvitis, or any combinations thereof. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor. In some embodiments, Ibrutinib and the CYP3A4 inhibitor are in a combined dosage form. In some embodiments, Ibrutinib and the CYP3A4 inhibitor are in separate dosage forms. In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially. In some embodiments, Ibrutinib is amorphous or crystalline.

Disclosed herein, in certain embodiments, are methods of treating a thromboembolic disorder in an individual in need thereof comprising administering a combination of a Btk inhibitor and a CYP3A4 inhibitor. Further disclosed herein, in certain embodiments, are methods of treating a thromboembolic disorder in an individual in need thereof comprising administering a combination of Ibrutinib and a CYP3A4 inhibitor. In some embodiments, the thromboembolic disorder is myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, the method increases the Cmax of Ibrutinib. In some embodiments, Cmax of Ibrutinib is increased by about 20× to about 40× the Cmax of Ibrutinib administered without a CYP3A4 inhibitor, or about 25× to about 35×. In some embodiments, the method increases the AUC of Ibrutinib. In some embodiments, the method increases the AUC of Ibrutinib by about 15× to about 35× the AUC of Ibrutinib administered without a CYP3A4 inhibitor, or about 20× to about 30×. In some embodiments, the method increases the AUC of Ibrutinib by about 2× to about 35× the AUC of Ibrutinib administered without a CYP3A4 inhibitor. In some embodiments, the method increases the AUC of Ibrutinib by about 2× to about 30× the AUC of Ibrutinib administered without a CYP3A4 inhibitor. In some embodiments, the method increases the AUC of Ibrutinib by about 2× to about 25× the AUC of Ibrutinib administered without a CYP3A4 inhibitor. In some embodiments, the method increases the AUC of Ibrutinib by about 2× to about 20× the AUC of Ibrutinib administered without a CYP3A4 inhibitor. In some embodiments, the method increases the AUC of Ibrutinib by about 2× to about 15× the AUC of Ibrutinib administered without a CYP3A4 inhibitor. In some embodiments, the method increases the AUC of Ibrutinib by about 2× to about 10× the AUC of Ibrutinib administered without a CYP3A4 inhibitor. In some embodiments, the method method increases the AUC of Ibrutinib by about 2× to about 5× the AUC of Ibrutinib administered without a CYP3A4 inhibitor. In some embodiments, the method increases the AUC of Ibrutinib by about 2× to about 4× the AUC of Ibrutinib administered without a CYP3A4 inhibitor. In some embodiments, the method does not significantly affect the Tmax or T1/2 of Ibrutinib as compared to the Tmax and T1/2 of Ibrutinib administered without a CYP3A4 inhibitor. In some embodiments, Ibrutinib and the CYP3A4 inhibitor are in a combined dosage form. In some embodiments, Ibrutinib and the CYP3A4 inhibitor are in separate dosage forms. In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially. In some embodiments, Ibrutinib is amorphous or crystalline.

### Additional Combination Therapies

In certain instances, it is appropriate to administer a Btk inhibitor and a CYP3A4 inhibitor in combination with an additional therapeutic agent. In certain instances, it is appropriate to administer Ibrutinib and a CYP3A4 inhibitor in combination with an additional therapeutic agent. Additional therapeutic agents are selected for their particular usefulness against the condition that is being treated. In general, the additional therapeutic agent does not need to be administered in the same pharmaceutical composition, at the same time or via the same route and the Ibrutinib and/or CYP3A4 inhibitor. In one embodiment, the initial administration is made according to established protocols, and then, based upon the observed effects, the dosage, modes of administration and times of administration, further modified.

In some embodiments, the additional therapeutic agent is administered concurrently (e.g., simultaneously, essentially simultaneously or within the same treatment protocol) or sequentially, depending upon the nature of the disease, the condition of the patient, and the actual choice of compounds used. In certain embodiments, the determination of the order of administration, and the number of repetitions of administration of each therapeutic agent during a treatment protocol, is based upon evaluation of the disease being treated and the condition of the patient.

The dose of the additional therapeutic agent varies depending on the additional therapeutic agent, the disease or condition being treated and so forth.

Disclosed herein, in certain embodiments, are methods of treating an autoimmune disorder, a heteroimmune disorder, an inflammatory disorder and/or a cancer in an individual in need thereof, comprising administering to the individual a Btk inhibitor, a CYP3A4 inhibitor, and an additional therapeutic agent. Further disclosed herein, in certain embodiments, are methods of treating an autoimmune disorder in an individual in need thereof, comprising administering to the individual a Btk inhibitor, a CYP3A4 inhibitor, and an additional therapeutic agent. Also disclosed herein, in certain embodiments, are methods of treating a heteroimmune disorder in an individual in need thereof comprising administering to the individual a Btk inhibitor, a CYP3A4 inhibitor, and an additional therapeutic agent. Disclosed herein, in certain embodiments, are methods of treating an inflammatory disorder in an individual in need thereof, comprising administering to the individual a Btk inhibitor, a CYP3A4 inhibitor, and an additional therapeutic agent. Further disclosed herein, in certain embodiments, are methods of treating a cancer in an individual in need thereof, comprising administering to the individual a Btk inhibitor, a CYP3A4 inhibitor, and an additional therapeutic agent.

Disclosed herein, in certain embodiments, are methods of treating an autoimmune disorder, a heteroimmune disorder, an inflammatory disorder and/or a cancer in an individual in need thereof, comprising administering to the individual Ibrutinib, a CYP3A4 inhibitor, and an additional therapeutic agent. Further disclosed herein, in certain embodiments, are methods of treating an autoimmune disorder in an individual in need thereof, comprising administering to the individual Ibrutinib, a CYP3A4 inhibitor, and an additional therapeutic agent. Also disclosed herein, in certain embodiments, are methods of treating a heteroimmune disorder in an individual in need thereof comprising administering to the individual Ibrutinib, a CYP3A4 inhibitor, and an additional therapeutic agent. Disclosed herein, in certain embodiments, are methods of treating an inflammatory disorder in an individual in need thereof, comprising administering to the individual Ibrutinib, a CYP3A4 inhibitor, and an additional therapeutic agent. Further disclosed herein, in certain embodiments, are methods of treating a cancer in an individual in need thereof, comprising administering to the individual Ibrutinib, a CYP3A4 inhibitor, and an additional therapeutic agent.

In some embodiments, administering a Btk inhibitor before a second cancer treatment regimen reduces immune-mediated reactions to the second cancer treatment regimen. In some embodiments, administering Ibrutinib before ofatumumab reduces immune-mediated reactions to ofatumumab.

In some embodiments, the additional therapeutic agent is a chemotherapeutic agent, a steroid, an immunotherapeutic agent, a targeted therapy, or a combination thereof. In some embodiments, the additional therapeutic agent is a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCy inhibitor, a PKCβ inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof

In some embodiments, the additional therapeutic agent is chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, l ibritumomab, tositumomab, bortezomib, pentostatin, endostatin, or a combination thereof.

In some embodiments, the additional therapeutic agent is cyclophosphamide, hydroxydaunorubicin, vincristine, and prednisone, and optionally, rituximab.

In some embodiments, the additional therapeutic agent is bendamustine, and rituximab.

In some embodiments, the additional therapeutic agent is fludarabine, cyclophosphamide, and rituximab.

In some embodiments, the additional therapeutic agent is cyclophosphamide, vincristine, and prednisone, and optionally, rituximab.

In some embodiments, the additional therapeutic agent is etoposide, doxorubicin, vincristine, cyclophosphamide, prednisolone, and optionally, rituximab.

In some embodiments, the additional therapeutic agent is dexamethasone and lenalidomide.

Additional therapeutic agents that maybe administered in conjunction with the combination of Ibrutinib and a CYP3A4 inhibitor include, but are not limited to, Nitrogen Mustards such as for example, bendamustine, chlorambucil, chlormethine, cyclophosphamide, ifosfamide, melphalan, prednimustine, trofosfamide; Alkyl Sulfonates like busulfan, mannosulfan, treosulfan; Ethylene Imines like carboquone, thiotepa, triaziquone; Nitrosoureas like carmustine, fotemustine, lomustine, nimustine, ranimustine, semustine, streptozocin; Epoxides such as for example, etoglucid; Other Alkylating Agents such as for example dacarbazine, mitobronitol, pipobroman, temozolomide; Folic Acid Analogues such as for example methotrexate, permetrexed, pralatrexate, raltitrexed; Purine Analogs such as for example cladribine, clofarabine, fludarabine, mercaptopurine, nelarabine, tioguanine; Pyrimidine Analogs such as for example azacitidine, capecitabine, carmofur, cytarabine, decitabine, fluorouracil, gemcitabine, tegafur; Vinca Alkaloids such as for example vinblastine, vincristine, vindesine, vinflunine, vinorelbine; Podophyllotoxin Derivatives such as for example etoposide, teniposide; Colchicine derivatives such as for example demecolcine; Taxanes such as for example docetaxel, paclitaxel, paclitaxel poliglumex; Other Plant Alkaloids and Natural Products such as for example trabectedin; Actinomycines such as for example dactinomycin; Antracyclines such as for example aclarubicin, daunorubicin, doxorubicin, epirubicin, idarubicin, mitoxantrone, pirarubicin, valrubicin, zorubincin; Other Cytotoxic Antibiotics such as for example bleomycin, ixabepilone, mitomycin, plicamycin; Platinum Compounds such as for example carboplatin, cisplatin, oxaliplatin, satraplatin; Methylhydrazines such as for example procarbazine; Sensitizers such as for example aminolevulinic acid, efaproxiral, methyl aminolevulinate, porfimer sodium, temoporfin; Protein Kinase Inhibitors such as for example dasatinib, erlotinib, everolimus, gefitinib, imatinib, lapatinib, nilotinib, pazonanib, sorafenib, sunitinib, temsirolimus; Other Antineoplastic Agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; Estrogens such as for example diethylstilbenol, ethinylestradiol, fosfestrol, polyestradiol phosphate; Progestogens such as for example gestonorone, medroxyprogesterone, megestrol; Gonadotropin Releasing Hormone Analogs such as for example buserelin, goserelin, leuprorelin, triptorelin; Anti-Estrogens such as for example fulvestrant, tamoxifen, toremifene; Anti-Androgens such as for example bicalutamide, flutamide, nilutamide, Enzyme Inhibitors, aminoglutethimide, anastrozole, exemestane, formestane, letrozole, vorozole; Other Hormone Antagonists such as for example abarelix, degarelix; Immunostimulants such as for example histamine dihydrochloride, mifamurtide, pidotimod, plerixafor, roquinimex, thymopentin; Immunosuppressants such as for example everolimus, gusperimus, leflunomide, mycophenolic acid, sirolimus; Calcineurin Inhibitors such as for example ciclosporin, tacrolimus; Other Immunosuppressants such as for example azathioprine, lenalidomide, methotrexate, thalidomide; and Radiopharmaceuticals such as for example, iobenguane.

Further therapeutic agents that maybe administered in conjunction with the combination of Ibrutinib and a CYP3A4 inhibitor include, but are not limited to interferons, interleukins, Tumor Necrosis Factors, Growth Factors, or the like.

Additional therapeutic agents that maybe administered in conjunction with the combination of Ibrutinib and a CYP3A4 inhibitor include, but are not limited to, Immunostimulants such as for example ancestim, filgrastim, lenograstim, molgramostim, pegfilgrastim, sargramostim; Interferons such as for example interferon alfa natural, interferon alfa-2a, interferon alfa-2b, interferon alfacon-1, interferon alfa-nl, interferon beta natural, interferon beta-1 a, interferon beta-1b, interferon gamma, peginterferon alfa-2a, peginterferon alfa-2b; Interleukins such as for example aldesleukin, oprelvekin; Other Immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; Calcineurin Inhibitors such as for example ciclosporin, tacrolimus; Other Immunosuppressants such as for example azathioprine, lenalidomide, methotrexate, thalidomide.

Further therapeutic agents that maybe administered in conjunction with the combination of Ibrutinib and a CYP3A4 inhibitor include, but are not limited to, Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, or the like, or a combination thereof.

Additional therapeutic agents that maybe administered in conjunction with the combination of Ibrutinib and a CYP3A4 inhibitor include, but are not limited to, Monoclonal Antibodies such as for example alemtuzumab, bevacizumab, catumaxomab, cetuximab, edrecolomab, gemtuzumab, ofatumumab, panitumumab, rituximab, trastuzumab, Immunosuppressants, eculizumab, efalizumab, muromab-CD3, natalizumab; TNF alpha Inhibitors such as for example adalimumab, afelimomab, certolizumab pegol, golimumab, infliximab, Interleukin Inhibitors, basiliximab, canakinumab, daclizumab, mepolizumab, tocilizumab, ustekinumab, Radiopharmaceuticals, ibritumomab tiuxetan, tositumomab; Others Monoclonal Antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

Further therapeutic agents that maybe administered in conjunction with the combination of Ibrutinib and a CYP3A4 inhibitor include, but are not limited to, agents that affect the tumor micro-environment such as cellular signaling network (e.g. phosphatidylinositol 3-kinase (PI3K) signaling pathway, signaling from the B-cell receptor and the IgE receptor). In some embodiments, the second agent is a PI3K signaling inhibitor or a syc kinase inhibitor. In one embodiment, the syk inhibitor is R788. In another embodiment is a PKCy inhibitor such as by way of example only, enzastaurin.

Examples of agents that affect the tumor micro-environment include PI3K signaling inhibitor, syc kinase inhibitor, Protein Kinase Inhibitors such as for example dasatinib, erlotinib, everolimus, gefitinib, imatinib, lapatinib, nilotinib, pazonanib, sorafenib, sunitinib, temsirolimus; Other Angiogenesis Inhibitors such as for example GT-111, JI-101, R1530; Other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, SCH 727965, SGI-1176, SGX523, SNS-314, TAK-593, TAK-901, TKI258, TLN-232, TTP607, XL147, XL228, XL281R05126766, XL418, XL765.

Further examples of therapeutic agents for use in combination with Ibrutinib and a CYP3A4 inhibitor include, but are not limited to, inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other agents that may be employed in combination with Ibrutinib and a CYP3A4 inhibitor include, but are not limited to, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or r1L2), interferon alfa-2a; interferon alfa-2b; interferon alfa-nl; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further therapeutic agents that maybe administered in conjunction with the combination of Ibrutinib and a CYP3A4 inhibitor include, but are not limited to, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1 -based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other therapeutic agents that maybe administered in conjunction with the combination of Ibrutinib and a CYP3A4 inhibitor include, but are not limited to, alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analogs (e.g., methotrexate), or pyrimidine analogs (e.g., Cytarabine), purine analogs (e.g., mercaptopurine, thioguanine, pentostatin).

Examples of alkylating agents that include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Additional therapeutic agents that maybe administered in conjunction with the combination of Ibrutinib and a CYP3A4 inhibitor include, but are not limited to,: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Ser.HCI, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (-)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the individual is suffering from or at risk of suffering from an autoimmune disease, an inflammatory disease, or an allergy disease, Ibrutinib and a CYP3A4 inhibitor may be used in combination with : immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β3, interferon-y, interleukin-2, allergy vaccines, antihistamines, antileukotrienes, beta-agonists, theophylline, or anticholinergics.

### Pharmaceutical Compositions/Formulations

Disclosed herein, in certain embodiments, are pharmaceutical compositions comprising (a) a Btk inhibitor and a CYP3A4 inhibitor, and (b) a pharmaceutically-acceptable excipient. Further disclosed herein, in certain embodiments, are pharmaceutical compositions comprising (a) Ibrutinib and a CYP3A4 inhibitor, and (b) a pharmaceutically-acceptable excipient. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, Ibrutinib is amorphous or crystalline. In some embodiments, Ibrutinib is milled or a nano-particle. In some embodiments, the pharmaceutical composition is a combined dosage form. In some embodiments, the composition increases the oral bioavailability of Ibrutinib. In some embodiments, the composition increases the Cmax of Ibrutinib. In some embodiments, the composition increases the AUC of Ibrutinib. In some embodiments, the composition increases the Cmax of Ibrutinib by about 20× to about 40× the Cmax of Ibrutinib administered without a CYP3A4 inhibitor, or about 25× to about 35×. In some embodiments, the composition increases the AUC of Ibrutinib by about 15× to about 35× the AUC of Ibrutinib administered without a CYP3A4 inhibitor, or about 20× to about 30×. In some embodiments, the composition comprises an amount of the CYP3A4 inhibitor that is effective to increase the AUC of Ibrutinib by about 2× to about 35× the AUC of Ibrutinib administered without a CYP3A4 inhibitor. In some embodiments, the composition comprises an amount of the CYP3A4 inhibitor that is effective to increase the AUC of Ibrutinib by about 2× to about 30× the AUC of Ibrutinib administered without a CYP3A4 inhibitor. In some embodiments, the composition comprises an amount of the CYP3A4 inhibitor that is effective to increase the AUC of Ibrutinib by about 2× to about 25× the AUC of Ibrutinib administered without a CYP3A4 inhibitor. In some embodiments, the composition comprises an amount of the CYP3A4 inhibitor that is effective to increase the AUC of Ibrutinib by about 2× to about 20× the AUC of Ibrutinib administered without a CYP3A4 inhibitor. In some embodiments, the composition comprises an amount of the CYP3A4 inhibitor that is effective to increase the AUC of Ibrutinib by about 2× to about 15× the AUC of Ibrutinib administered without a CYP3A4 inhibitor. In some embodiments, the composition comprises an amount of the CYP3A4 inhibitor that is effective to increase the AUC of Ibrutinib by about 2× to about 10× the AUC of Ibrutinib administered without a CYP3A4 inhibitor. In some embodiments, the composition comprises an amount of the CYP3A4 inhibitor that is effective to increase the AUC of Ibrutinib by about 2× to about 5× the AUC of Ibrutinib administered without a CYP3A4 inhibitor. In some embodiments, the composition comprises an amount of the CYP3A4 inhibitor that is effective to increase the AUC of Ibrutinib by about 2× to about 4× the AUC of Ibrutinib administered without a CYP3A4 inhibitor. In some embodiments, the composition does not significantly affect the Tmax or T1/2 of Ibrutinib as compared to the Tmax and T1/2 of Ibrutinib administered without a CYP3A4 inhibitor. In some embodiments, the pharmaceutical compositions further comprise chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the pharmaceutical compositions further comprise cyclophosphamide, hydroxydaunorubicin, vincristine, and prednisone, and optionally, rituximab. In some embodiments, the pharmaceutical compositions further comprise bendamustine, and rituximab. In some embodiments, the pharmaceutical compositions further comprise fludarabine, cyclophosphamide, and rituximab. In some embodiments, the pharmaceutical compositions further comprise cyclophosphamide, vincristine, and prednisone, and optionally, rituximab. In some embodiments, the pharmaceutical compositions further comprise etoposide, doxorubicin, vincristine, cyclophosphamide, prednisolone, and optionally, rituximab. In some embodiments, the pharmaceutical compositions further comprise dexamethasone and lenalidomide.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

A pharmaceutical composition, as used herein, refers to a mixture of Ibrutinib, a CYP3A4 inhibitor, and/or an additional therapeutic agent with other chemical components, such as carriers, stabilizers, diluents, dispersing agents, suspending agents, thickening agents, and/or excipients.

In practicing the methods of treatment or use provided herein, therapeutically effective amounts of the compounds disclosed herein are administered having a disease, disorder, or condition to be treated. In some embodiments, the mammal is a human. The therapeutically effective amounts of the compounds may vary depending on the compounds, severity of the disease, the age and relative health of the subject, and other factors.

The term “combination” as used herein, means a product that results from the mixing or combining of Ibrutinib and a CYP3A4 inhibitor (and any additional therapeutic agents) and includes both fixed and non-fixed combinations. The term “fixed combination” means that Ibrutinib and the CYP3A4 inhibitor are both administered in a single entity or dosage form. The term “non-fixed combination” means that Ibrutinib and the CYP3A4 inhibitor are administered as separate entities or dosage forms either simultaneously, concurrently or sequentially with no specific intervening time limits, wherein such administration provides effective levels of the two compounds in the body of the patient. The latter also applies to cocktail therapy, e.g. the administration of three or more active ingredients.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

### Dosage Forms

Disclosed herein, in certain embodiments, are dosage forms comprising a Btk inhibitor and a CYP3A4 inhibitor. Further disclosed herein, in certain embodiments, are dosage forms comprising Ibrutinib and a CYP3A4 inhibitor. In some embodiments, the dosage form is a combined dosage form. In some embodiments, the dosage form is a solid oral dosage form. In some embodiments, the dosage form is a tablet, pill, or capsule. In some embodiments, the dosage form is a controlled release dosage form, delayed release dosage form, extended release dosage form, pulsatile release dosage form, multiparticulate dosage form, or mixed immediate release and controlled release formulation. In some embodiments, the dosage form comprises a controlled release coating. In some embodiments, the dosage forms comprises a first controlled release coating which controls the release of Ibrutinib and a second controlled release coating which controls the release of the CYP3A4 inhibitor. In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin, verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the dose of Ibrutinib is between about 10 mg to about 100 mg. In some embodiments, the therapeutically-effective amount of Ibrutinib is between about 40 mg and about 100 mg. In some embodiments, the dose of Ibrutinib is between about 40 mg and about 70 mg. In some embodiments, the dose of Ibrutinib is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the dose of Ibrutinib is about 40 mg. In some embodiments, Ibrutinib is amorphous or crystalline. In some embodiments, the dosage form increases the oral bioavailability of Ibrutinib. In some embodiments, the dosage form increases the Cmax of Ibrutinib. In some embodiments, the dosage form increases the AUC of Ibrutinib. In some embodiments, the dosage form increases the Cmax of Ibrutinib by about 20× to about 40× the Cmax of Ibrutinib administered without a CYP3A4 inhibitor, or about 25× to about 35×. In some embodiments, the dosage form increases the AUC of Ibrutinib by about 15× to about 35× the AUC of Ibrutinib administered without a CYP3A4 inhibitor, or about 20× to about 30×. In some embodiments, the dosage form increases the AUC of Ibrutinib by about 2× to about 35× the AUC of Ibrutinib administered without a CYP3A4 inhibitor. In some embodiments, the dosage form increases the AUC of Ibrutinib by about 2× to about 30× the AUC of Ibrutinib administered without a CYP3A4 inhibitor. In some embodiments, the dosage form increases the AUC of Ibrutinib by about 2× to about 25× the AUC of Ibrutinib administered without a CYP3A4 inhibitor. In some embodiments, the dosage form increases the AUC of Ibrutinib by about 2× to about 20× the AUC of Ibrutinib administered without a CYP3A4 inhibitor. In some embodiments, the dosage form increases the AUC of Ibrutinib by about 2× to about 15× the AUC of Ibrutinib administered without a CYP3A4 inhibitor. In some embodiments, the dosage form increases the AUC of Ibrutinib by about 2× to about 10× the AUC of Ibrutinib administered without a CYP3A4 inhibitor. In some embodiments, the dosage form increases the AUC of Ibrutinib by about 2× to about 5× the AUC of Ibrutinib administered without a CYP3A4 inhibitor. In some embodiments, the dosage form increases the AUC of Ibrutinib by about 2× to about 4× the AUC of Ibrutinib administered without a CYP3A4 inhibitor. In some embodiments, the dosage form does not significantly affect the Tmax or T1/2 of Ibrutinib as compared to the Tmax and T1/2 of Ibrutinib administered without a CYP3A4 inhibitor. In some embodiments, the dosage forms further comprise chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, l ibritumomab, tositumomab, bortezomib, pentostatin, endostatin, or a combination thereof. In some embodiments, the dosage forms further comprise cyclophosphamide, hydroxydaunorubicin, vincristine, and prednisone, and optionally, rituximab. In some embodiments, the dosage forms further comprise bendamustine, and rituximab. In some embodiments, the dosage forms further comprise fludarabine, cyclophosphamide, and rituximab. In some embodiments, the dosage forms further comprise cyclophosphamide, vincristine, and prednisone, and optionally, rituximab. In some embodiments, the dosage forms further comprise etoposide, doxorubicin, vincristine, cyclophosphamide, prednisolone, and optionally, rituximab. In some embodiments, the dosage forms further comprise dexamethasone and lenalidomide.

The pharmaceutical compositions described herein may be formulated for administration via any conventional means including, but not limited to, oral, parenteral (e.g., intravenous, subcutaneous, or intramuscular), buccal, intranasal, rectal or transdermal administration routes. As used herein, the terms “subject”, “individual” and “patient” are used interchangeably and mean an animal, preferably a mammal, including a human or non-human. None of the terms require the supervision (continuous or otherwise) of a medical professional.

The pharmaceutical compositions described herein are formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical dosage forms described herein may include one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the pharmaceutical compositions. Dosing and Treatment Regimens

In some embodiments, the amount of Ibrutinib that is administered in combination with a CYP3A4 inhibitor is from 40 mg/day up to, and including, 1000 mg/day. In some embodiments, the amount of Ibrutinib that is administered is from about 40 mg/day to 70 mg/day. In some embodiments, the amount of Ibrutinib that is administered per day is about 10, mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of Ibrutinib that is administered is about 40 mg/day. In some embodiments, the amount of Ibrutinib that is administered is about 50 mg/day. In some embodiments, the amount of Ibrutinib that is administered is about 60 mg/day. In some embodiments, the amount of Ibrutinib that is administered is about 70 mg/day.

In some embodiments, the AUCO-24 of Ibrutinib co-administered with a CYP3A4 inhibitor is between about 50 and about 10000 ng*h/mL. In some embodiments, the Cmax of Ibrutinib co-administered with a CYP3A4 inhibitor is between about 5 ng/mL and about 1000 ng/mL.

In some embodiments, Ibrutinib is administered once per day, twice per day, or three times per day. In some embodiments, Ibrutinib is administered once per day. In some embodiments, the CYP3A4 inhibitor is administered once per day, twice per day, or three times per day. In some embodiments, the CYP3A4 inhibitor is administered once per day. In some embodiments, Ibrutinib and the CYP3A4 inhibitor are co-administered (e.g., in a single dosage form), once per day. In some embodiments, Ibrutinib and the CYP3A4 inhibitor are maintenance therapy.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

In some embodiments, the Btk inhibitor and the CYP3A4 inhibitor are administered concurrently. In some embodiments, the Btk inhibitor and the CYP3A4 inhibitor are administered simultaneously, essentially simultaneously or within the same treatment protocol. In some embodiments, the Btk inhibitor and the CYP3A4 inhibitor are administered sequentially.

In some embodiments, Ibrutinib and the CYP3A4 inhibitor are administered concurrently. In some embodiments, Ibrutinib and the CYP3A4 inhibitor are administered simultaneously, essentially simultaneously or within the same treatment protocol. In some embodiments, Ibrutinib and the CYP3A4 inhibitor are administered sequentially.

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include Ibrutinib, optionally in a composition or in combination with a CYP3A4 inhibitor as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The following ingredients, formulations, processes and procedures for practicing the methods disclosed herein correspond to that described above.

### Example 1: Study to Assess the Effects of Ketoconazole on the Pharmacokinetics of Ibrutinib in Healthy Subiects

Purpose: The purpose of this study is to establish the effects of ketoconazole on the pharmacokinetics of orally administered Ibrutinib.

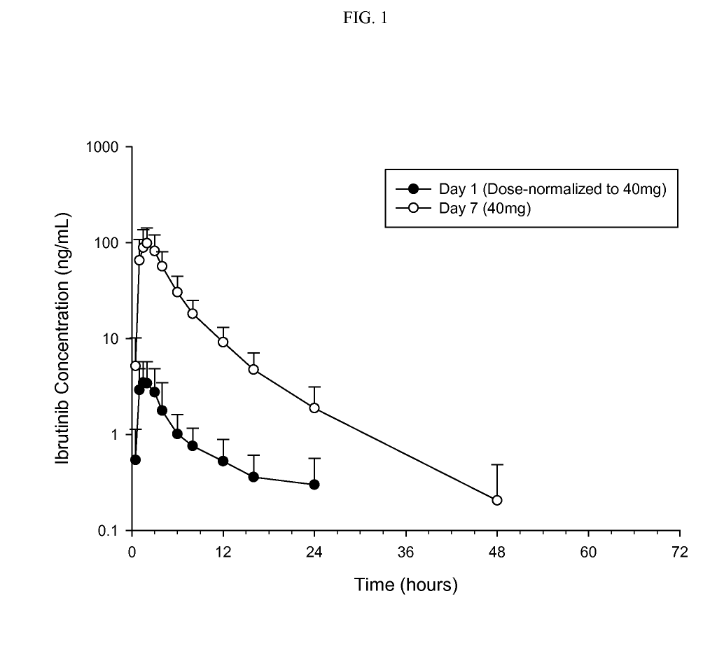

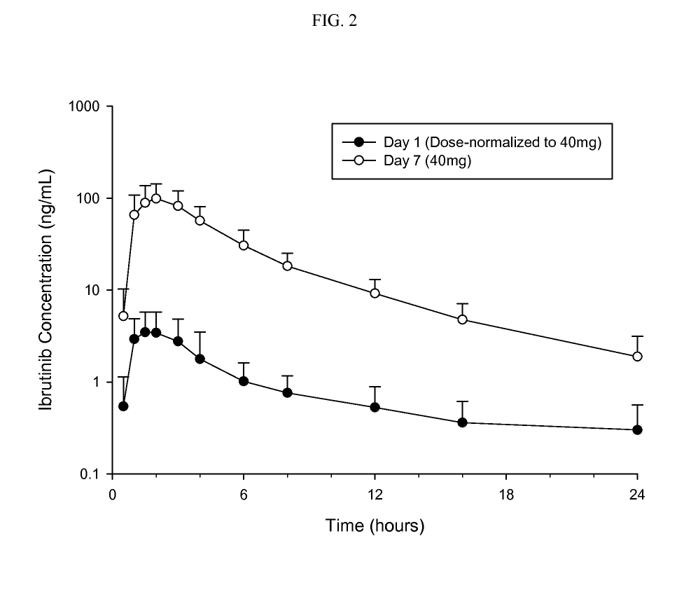

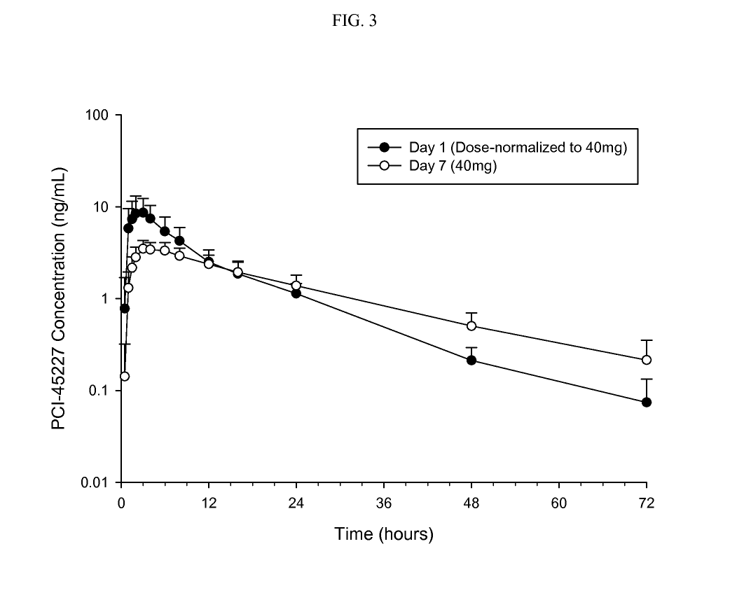

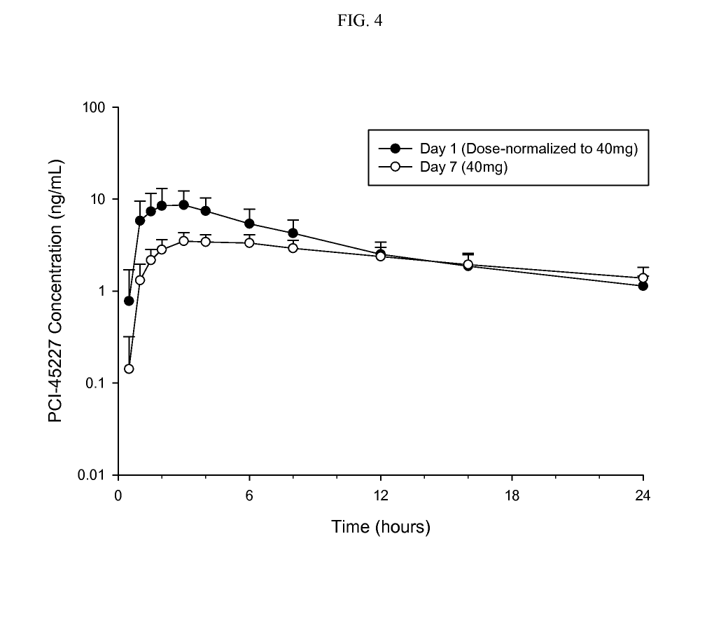

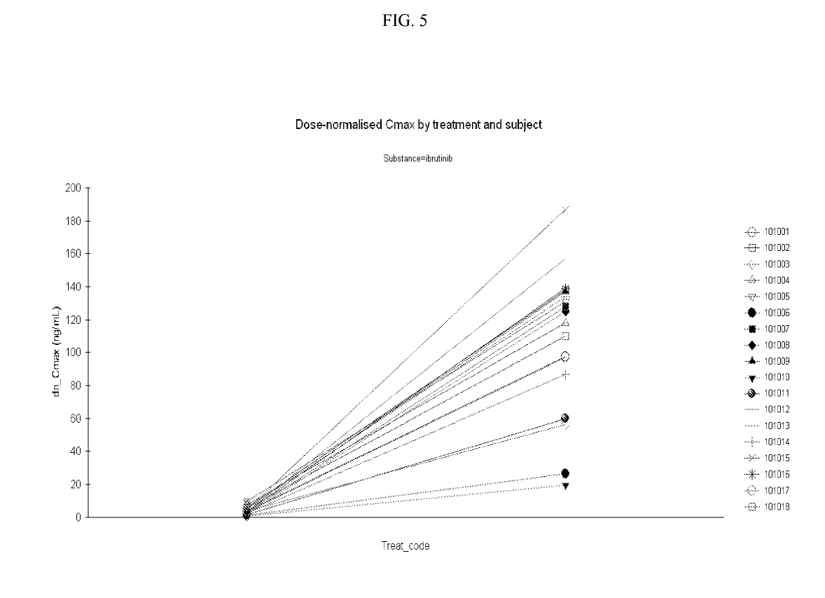

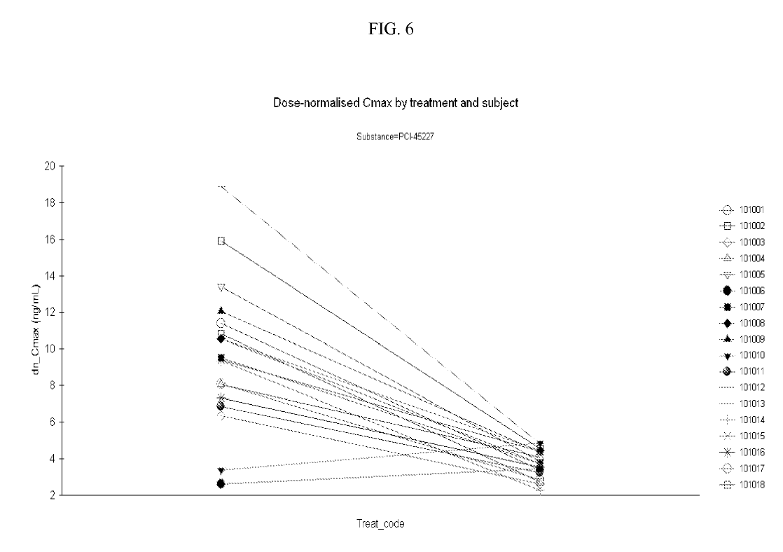

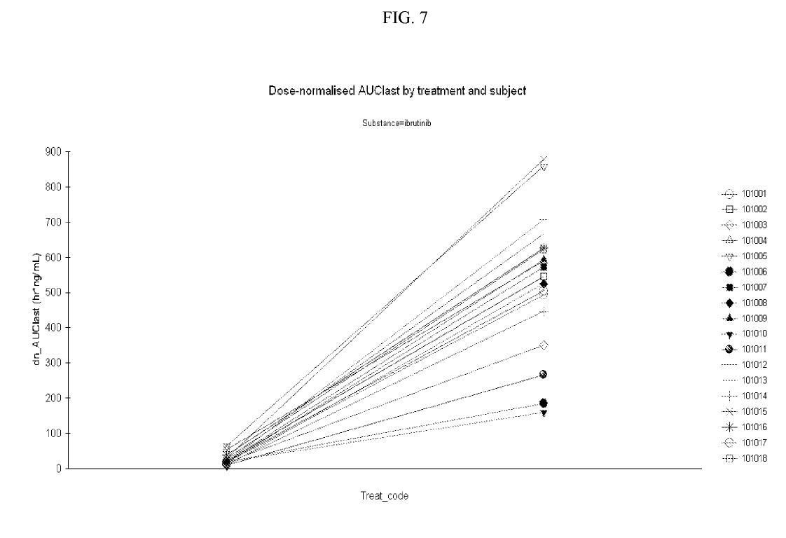

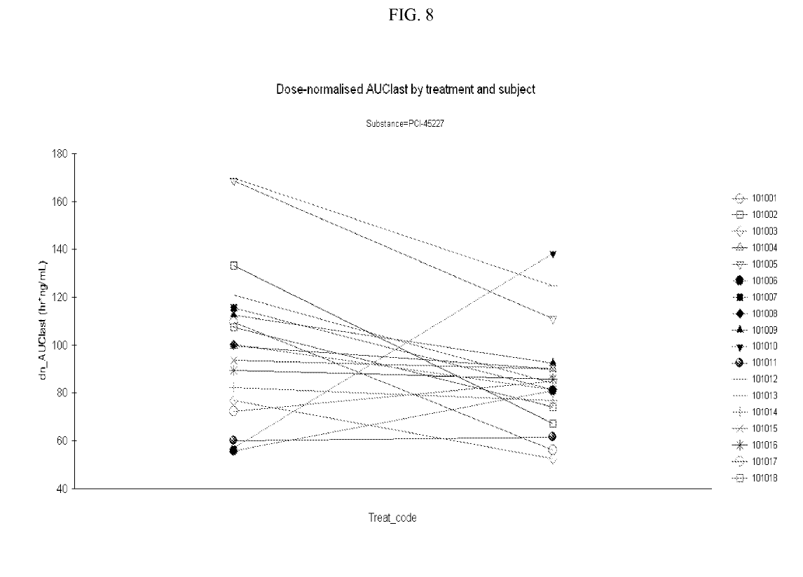

18 healthy male subjects were recruited. They received 120 mg of Ibrutinib (3×40 mg) alone on Day 1, and 40 mg of Ibrutinib in combination with ketoconazole on Day 7. Ketoconazole (400 mg [2×200 mg] once daily) was orally administered alone on Days 4 to 6, hour prior to Ibrutinib dosing on Day 7, and alone again on Days 8 and 9. Full pK was measured to 72 hours. The study shows that Ibrutinib systemic exposure in healthy subjects is significantly affected when dosed concomitantly with ketoconazole. The results are presented in FIGS. 1, 2, 5, and 7, and Tables 1 a (pK parameters Day 1, Ibrutinib), lb (pK parameters Day 1, Ibrutinib), 3a (pK parameters Day 7, Ibrutinib), 3b (pK parameters Day 7, Ibrutinib), 5 (Ibrutinib pK parameters, Day 1) and 6 (Ibrutinib pK parameters, Day 7). Results of co-administration of ketoconazole on the pharmacokinetics of PCI-45227 are presented in FIGS. 3, 4, 6 and 8, and Tables 2a (pK parameters Day 1, PCI-45227), 2b (pK parameters Day 1, PCI-45227), 4a (pK parameters Day 7, PCI-45227), 4b (pK parameters Day 7, PCI-45227), 7 (PCI-45227 pK parameters, Day 1), and 8 (PCI-45227 pK parameters, Day 7).

TABLE 1a
Ibrutinib (Plasma)

| | DN*Cmax | Tmax | tlast | DN*AUC0-24 | DN*AUClast |
|---|---|---|---|---|---|
| | (ng/mL) | (h) | (h) | (h · ng/mL) | (h · ng/mL) |
| n | 18 | 18 | 18 | 18 | 18 |
| Mean | 3.92 | 1.89 | 34.00 | 21.3 | 23.8 |
| SD | 2.22 | 0.70 | 15.54 | 12.4 | 15.0 |
| % CV | 56.8 | 37.2 | 45.7 | 58.4 | 63.1 |
| Median | 3.57 | 1.75 | 24.01 | 18.5 | 19.2 |
| Min | 0.923 | 1.00 | 12.00 | 7.55 | 7.47 |
| Max | 9.67 | 3.03 | 72.00 | 55.8 | 64.0 |
| Geom. | 3.33 | 1.77 | 30.93 | 18.7 | 20.4 |
| mean | | | | | |

TABLE 1b
Ibrutinib (Plasma)

| | DN* | | | | | | |
|---|---|---|---|---|---|---|---|
| | AUC∞ | T½ | | AUMC∞ | | | |
| | (h · ng/ | term | λz | (h · h · | MRT | Vd/F | CL/F |
| | mL) | (h) | (1/h) | ng/mL) | (h) | (L) | (L/h) |
| n | 12 | 12 | 12 | 12 | 12 | 12 | 12 |
| Mean | 28.1 | 8.20 | 0.112 | 857 | 8.93 | 19049 | 2014 |
| SD | 17.5 | 3.22 | 0.0896 | 720 | 4.01 | 6133 | 1300 |
| % CV | 62.4 | 39.3 | 79.8 | 83.9 | 44.9 | 32.2 | 64.5 |
| Median | 22.4 | 8.63 | 0.0806 | 693 | 9.13 | 19027 | 1799 |
| Min | 7.58 | 1.85 | 0.0562 | 78.3 | 3.45 | 7189 | 617 |
| Max | 64.8 | 12.34 | 0.375 | 2117 | 17.8 | 28835 | 5279 |
| Geom. | 23.7 | 7.36 | 0.0941 | 576 | 8.11 | 17959 | 1691 |
| mean | | | | | | | |

TABLE 2a
PCI-45227 (Plasma)

| | DN*Cmax | Tmax | tlast | DN*AUC0-24 | DN*AUClast |
|---|---|---|---|---|---|
| | (ng/mL) | (h) | (h) | (h · ng/mL) | (h · ng/mL) |
| n | 18 | 18 | 18 | 18 | 18 |
| Mean | 9.70 | 2.42 | 66.7 | 82.2 | 101 |
| SD | 3.98 | 0.88 | 10.26 | 30.6 | 33.3 |
| % CV | 41.1 | 36.4 | 15.4 | 37.2 | 32.8 |
| Median | 9.45 | 2.00 | 72.00 | 81.3 | 100 |
| Min | 2.59 | 1.00 | 48.00 | 33.1 | 55.9 |
| Max | 18.9 | 4.00 | 72.05 | 144 | 170 |
| Geom. | 8.81 | 2.25 | 65.80 | 76.6 | 96.4 |
| mean | | | | | |

TABLE 2b
PCI-45227 (Plasma)

| | DN* | | | | | | |
|---|---|---|---|---|---|---|---|
| | AUC∞ | T½ | | AUMC∞ | | | |
| | (h · ng/ | term | λz | (h · h · | MRT | Vd/F | CL/F |
| | mL) | (h) | (1/h) | ng/mL) | (h) | (L) | (L/h) |
| n | 18 | 18 | 18 | 18 | 18 | 18 | 18 |
| Mean | 103 | 11.41 | 0.0630 | 4297 | 14.3 | 7073 | 428 |
| SD | 33.5 | 2.33 | 0.0119 | 1286 | 3.5 | 2960 | 140 |
| % CV | 32.5 | 20.4 | 18.8 | 29.9 | 24.4 | 41.9 | 32.8 |
| Median | 101 | 10.74 | 0.0645 | 3979 | 13.6 | 7059 | 395 |
| Min | 56.4 | 8.59 | 0.0412 | 2333 | 9.9 | 3428 | 231 |
| Max | 173 | 16.84 | 0.0807 | 6841 | 23.9 | 16146 | 709 |
| Geom. | 98.2 | 11.2 | 0.0619 | 4122 | 14.0 | 6578 | 407 |
| mean | | | | | | | |

TABLE 3a
Ibrutinib (Plasma)

| | Cmax | tmax | tlast | AUC0-24 | AUClast |
|---|---|---|---|---|---|
| | (ng/mL) | (h) | (h) | (h · ng/mL) | (h · ng/mL) |
| n | 18 | 18 | 18 | 18 | 18 |
| Mean | 108 | 1.97 | 42.68 | 510 | 533 |
| SD | 44.3 | 0.53 | 13.15 | 194 | 199 |
| % CV | 41.0 | 26.9 | 30.8 | 38.1 | 37.3 |
| Median | 122 | 2.00 | 48.00 | 541 | 559 |
| Min | 19.7 | 1.50 | 24.00 | 138 | 160 |
| Max | 187 | 3.03 | 72.00 | 878 | 878 |
| Geom. | 95.2 | 1.91 | 40.51 | 464 | 488 |
| mean | | | | | |

TABLE 3b
Ibrutinib (Plasma)

| | AUC∞ | T½ | | AUMC∞ | | | |
|---|---|---|---|---|---|---|---|
| | (h · ng/ | term | λz | (h · h · | MRT | Vd/F | CL/F |
| | mL) | (h) | (1/h) | ng/mL) | (h) | (L) | (L/h) |
| n | 18 | 18 | 18 | 18 | 18 | 18 | 18 |
| Mean | 536 | 6.32 | 0.124 | 3504 | 6.8 | 885 | 92.0 |
| SD | 199 | 2.00 | 0.0529 | 1531 | 2.0 | 729 | 55.8 |
| % CV | 37.2 | 31.6 | 42.5 | 43.7 | 29.0 | 82.3 | 60.7 |
| Median | 560 | 6.68 | 0.104 | 3635 | 6.2 | 708 | 71.5 |
| Min | 162 | 2.76 | 0.0708 | 1504 | 4.1 | 184 | 45.4 |
| Max | 882 | 9.79 | 0.251 | 8341 | 11.1 | 2812 | 247 |
| Geom. | 491 | 5.98 | 0.116 | 3235 | 6.6 | 702 | 81.4 |
| mean | | | | | | | |

TABLE 4a
PCI-45227 (Plasma)

| | Cmax | tmax | tlast | AUC0-24 | AUClast |
|---|---|---|---|---|---|
| | (ng/mL) | (h) | (h) | (h · ng/mL) | (h · ng/mL) |
| n | 18 | 18 | 18 | 18 | 18 |
| Mean | 3.71 | 3.95 | 68.01 | 54.5 | 85.2 |
| SD | 0.775 | 1.26 | 9.20 | 11.8 | 22.0 |
| % CV | 20.9 | 31.9 | 13.5 | 21.7 | 25.8 |
| Median | 3.74 | 4.00 | 72.00 | 53.8 | 82.7 |
| Min | 2.27 | 2.00 | 48.02 | 37.7 | 52.7 |
| Max | 4.86 | 6.02 | 72.05 | 83.2 | 138 |
| Geom. | 3.63 | 3.77 | 67.30 | 53.4 | 82.7 |
| mean | | | | | |

TABLE 4b
PCI-45227 (Plasma)

| | AUC∞ | T½ | | AUMC∞ | | | |
|---|---|---|---|---|---|---|---|
| | (h · ng/ | term | λz | (h · h · | MRT | Vd/F | CL/F |
| | mL) | (h) | (1/h) | ng/mL) | (h) | (L) | (L/h) |
| n | 18 | 18 | 18 | 18 | 18 | 18 | 18 |
| Mean | 92.2 | 17.99 | 0.0407 | 2416 | 25.4 | 11568 | 461 |
| SD | 24.8 | 4.57 | 0.00976 | 1069 | 5.7 | 2551 | 115 |
| % CV | 26.9 | 25.4 | 24.0 | 44.2 | 22.6 | 22.1 | 25.0 |
| Median | 89.6 | 16.61 | 0.0417 | 2177 | 24.8 | 11182 | 447 |
| Min | 58.3 | 10.56 | 0.0244 | 1073 | 15.6 | 6346 | 272 |
| Max | 147 | 28.43 | 0.0656 | 5288 | 40.6 | 17280 | 686 |
| Geom. | 89.4 | 17.49 | 0.0396 | 2216 | 24.8 | 11295 | 448 |
| mean | | | | | | | |

TABLE 5
Ibrutinib (Plasma)

| | | DN* | | | | DN* | |
|---|---|---|---|---|---|---|---|
| | | Cmax | | | AUC0-24 | AUC0-24 | T½, |
| | Cmax | (ng/ | tmax | tlast | (h · ng/ | (h · ng/ | term |
| | (ng/mL) | mL) | (h) | (h) | mL) | mL) | (h) |
| n | 18 | 18 | 18 | 18 | 18 | 18 | 12 |
| Mean | 11.8 | 3.92 | 1.89 | 34.00 | 63.8 | 21.3 | 8.20 |
| % CV | 56.8 | 56.8 | 37.2 | 45.7 | 58.4 | 58.4 | 39.3 |

TABLE 6
Ibrutinib (Plasma)

| | Cmax | tmax | tlast | AUC0-24 | tterm |
|---|---|---|---|---|---|
| | (ng/mL) | (h) | (h) | (h · ng/mL) | (h) |
| n | 18 | 18 | 18 | 18 | 12 |
| Mean | 108 | 1.97 | 42.68 | 510 | 6.32 |
| % CV | 41.0 | 26.9 | 30.8 | 38.1 | 31.6 |

TABLE 7
PCI-45227 (Plasma)

| | | DN* | | | | DN* | |
|---|---|---|---|---|---|---|---|
| | | Cmax | | | AUC0-24 | AUC0-24 | T½, |
| | Cmax | (ng/ | tmax | tlast | (h · ng/ | (h · ng/ | term |
| | (ng/mL) | mL) | (h) | (h) | mL) | mL) | (h) |
| n | 18 | 18 | 18 | 18 | 18 | 18 | 12 |
| Mean | 29.1 | 9.70 | 2.42 | 66.67 | 247 | 82.2 | 11.41 |
| % CV | 41.1 | 41.1 | 36.4 | 15.4 | 37.2 | 37.2 | 20.4 |

TABLE 8
PCI-45227 (Plasma)

| | | | | | T½, |
|---|---|---|---|---|---|
| | Cmax | tmax | tlast | AUC0-24 | term |
| | (ng/mL) | (h) | (h) | (h · ng/mL) | (h) |
| n | 18 | 18 | 18 | 18 | 12 |
| Mean | 3.71 | 3.95 | 68.01 | 54.5 | 17.99 |
| % CV | 20.9 | 31.9 | 13.5 | 21.7 | 25.4 |

### Example 2: Study to Assess the Effects of Grapefruit Juice on the Pharmacokinetics of Ibrutinib in Healthy Subjects

Purpose: The purpose of this study is to establish the effects of grapefruit juice on the pharmacokinetics of orally administered Ibrutinib.

8 healthy subjects were recruited for this cross-over study. They received 560 mg of Ibrutinib alone on Day 1. Seven days later, subjects were randomized into two arms; arm 1: 560 mg of Ibrutinib followed by a standard breakfast 30 minutes after dosing; and arm 2: subjects drank 240 mL of grapefruit juice the evening before and again 30 minutes before dosing Ibrutinib (140 mg) followed by a standard breakfast 30 minutes after dosing.

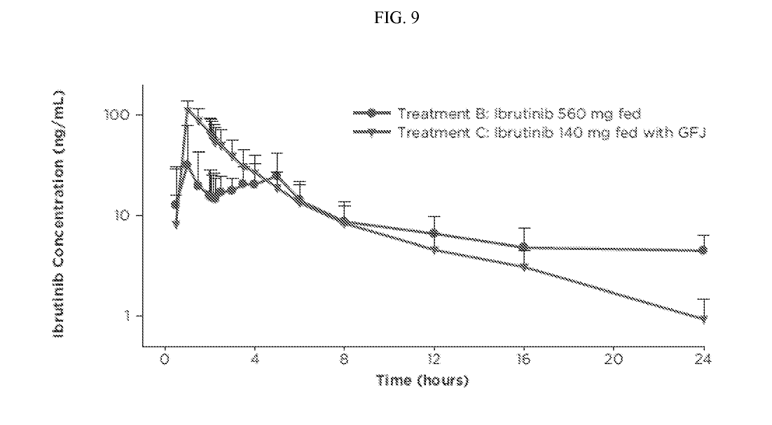

The study shows that food blunts the impact of the grapefruit juice, an intestinal CYP3A4 inhibitor, by increasing the mesenteric and splanchnic blood flow, resulting in higher systemic bioavailability compared to the fasted condition. Thus the effect on AUC caused by grapefruit juice in this study is less than estimated in the fasted condition. The results are presented in FIG. 9 and Table 9.

TABLE 9

| | Ibrutinib | Ibrutinib | Ibrutinib |
|---|---|---|---|
| | (560 mg) | (560 mg) | (140 mg) + |
| | Alone Fasted | Alone Fed | Grapefruit Juice |
| n | 8 | 8 | 8 |
| Tmax h, | 4 (1-4) | 2 (2-5) | 2 (1-2) |
| median (range) | | | |
| Cmax, ng/mL | 245 (43) | 128 (46) | 125 (68) |
| AUC_{0-24 h}, | 229 (107) | 544 (161)^{#} | 339 (97)^{#} |
| ng*h/mL | | | |
| AUCₗₐₛₜ, | 289 (117) | 606 (160) | 325 (103) |
| ng*h/mL | | | |
| AUC_{inf}, | 368 (80) | 659 (132) | 334 (105) |
| ng*h/mL | | | |
| T_{1/2}, h | 13 (5)^{##} | 10 (4)^{#} | 6 (2) |

^{#}n = 7;
^{##}n = 3

### Example 3: Study to Assess the Effects of Rifampin on the Pharmacokinetics of Ibrutinib in Healthy Subiects

Purpose: The purpose of this study is to establish the effects of rifampin, a CYP3A4 inducer, on the pharmacokinetics of orally administered Ibrutinib.

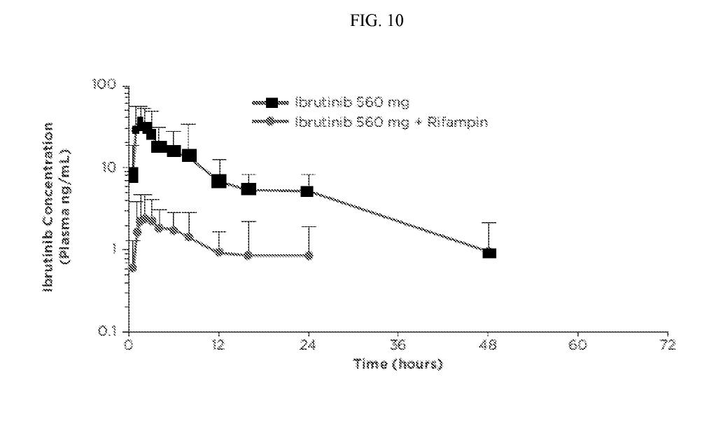

18 healthy subjects were recruited. They received a single oral dose of 560 mg of Ibrutinib (3×40 mg) alone on Day 1, and a single oral dose of 560 mg of Ibrutinib in combination with a single oral dose of 600 mg of rifampin on Day 11. Rifampin (600 mg once daily) was orally administered alone on Days 7 to 13. Serial blood samples for PK analysis of Ibrutinib were collected before dosing and over 72 hours following both Ibrutinib doses. The study shows that Ibrutinib systemic exposure in healthy subjects is significantly affected when dosed concomitantly with rifampin. The results are presented in FIG. 10 and Table 10.

TABLE 10

| | Ibrutinib (560 mg) | Ibrutinib (560 mg) + |
|---|---|---|
| | Alone | Rifampin |
| n | 18 | 17 |
| Tmax h, | 2 (1-8) | 3 (2-24) |
| median (range) | | |
| Cmax, ng/mL | 42 (30) | 3 (3) |
| AUC_{0-24 h}, | 259 (176) | 29 (23) |
| ng*h/mL | | |
| AUCₗₐₛₜ, | 335 (229) | 38 (37) |
| ng*h/mL | | |
| AUC_{inf}, | 397 (252)^{#} | 59 (64)^{##} |
| ng*h/mL | | |
| T_{1/2}, h | 10 (3)^{#} | 8 (4)^{##} |

^{#}n = 11;
^{##}n = 5

### Example 4: Metabolite (PCI-45227) to Ibrutinib Ratios of Studies from Examples 1-3

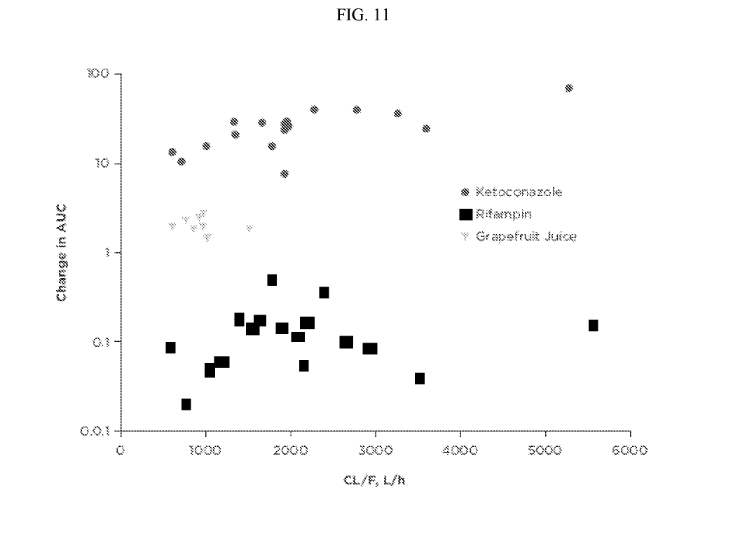

The metabolite (PCI-45227)/Ibrutnib ratios for the studies in Examples 1, 2, and 3 are shown in Tables 11, 12, and 13 respectively. The change in AUC versus baseline apparent clearance following oral administration of Ibrutinib with Ketoconazole, grapefruit juice, and Rifampin are shown in FIG. 11.

TABLE 11
Metabolite/Ibrutinib Ratios

| | |
|---|---|
| Ibrutinib Alone | Ibrutinib + Ketoconazole |

| | Cₘₐₓ | AUCₗₐₛₜ | Cₘₐₓ | AUCₗₐₛₜ |
|---|---|---|---|---|
| n | 18 | 18 | 18 | 18 |
| Mean | 2.64 | 5.03 | 0.0462 | 0.189 |
| % CV | 36.6 | 55.2 | 110.6 | 89.2 |
| Min | 1.00 | 1.70 | 0.0219 | 0.0952 |
| Max | 4.36 | 12.50 | 0.229 | 0.804 |

TABLE 12
Metabolite/Ibrutinib Ratios

| | | | | | Ibrutinib + | |
|---|---|---|---|---|---|---|
| | Ibrutinib Fasted | | Ibrutinib Fed | | Grapefruit Juice | |
| | Cₘₐₓ | AUCₗₐₛₜ | Cₘₐₓ | AUCₗₐₛₜ | Cₘₐₓ | AUCₗₐₛₜ |
| n | 8 | 8 | 8 | 8 | 8 | 8 |
| Mean | 2.41 | 3.09 | 1.03 | 1.95 | 0.317 | 0.836 |
| % CV | 46.3 | 42.8 | 29.8 | 34.1 | 45.7 | 41.3 |
| Min | 0.847 | 1.70 | 0.701 | 1.11 | 0.130 | 0.352 |
| Max | 4.60 | 5.86 | 1.65 | 3.03 | 0.544 | 1.23 |

TABLE 13
Metabolite/Ibrutinib Ratios

| | |
|---|---|
| Ibrutinib Alone | Ibrutinib + Rifampin |

| | Cₘₐₓ | AUCₗₐₛₜ | Cₘₐₓ | AUCₗₐₛₜ |
|---|---|---|---|---|
| n | 18 | 18 | 17 | 17 |
| Mean | 2.09 | 3.10 | 20.8 | 15.5 |
| % CV | 46.1 | 32.5 | 57.2 | 65.8 |
| Min | 0.70 | 1.57 | 2.23 | 1.31 |
| Max | 4.35 | 5.48 | 47.0 | 35.7 |

### Example 5: Safety and Tolerability Study of Co-Administration of Ibrutinib and GS9350 in Chronic Lymphocytic Leukemia

Purpose: The purpose of this study is to establish the safety and optimal dose of orally administered Ibrutinib and orally administered GS9350 in patients with B-cell chronic lymphocytic leukemia/small lymphocytic lymphoma/diffuse well-differentiated lymphocytic lymphoma.

Primary Outcome Measures:

Safety and tolerability of combination of Ibrutinib and GS9350 (frequency, severity, and relatedness of adverse events).

Secondary Outcome Measures:

Pharmacokinetic/Pharmacodynamic assessments.

Tumor response—overall response rate as defined by recent guidelines on CLL and SLL (B cell lymphoma) and duration of response.

Eligibility:

18 Years and older; both genders are eligible.

Inclusion Criteria:

For treatment-naive group only: Men and women >65 years of age with confirmed diagnosis of CLL/SLL, who require treatment per NCI or International Working Group guidelines 11-14.

For relapsed/refractory group only: Men and women >18 years of age with a confirmed diagnosis of relapsed/refractory CLL/SLL unresponsive to therapy (ie, failed >2 previous treatments for CLL/SLL and at least 1 regimen had to have had a purine analog [eg, fludarabine] for subjects with CLL).

Body weight >40 kg.

ECOG performance status of <2.

Agreement to use contraception during the study and for 30 days after the last dose of study drug if sexually active and able to bear children.

Willing and able to participate in all required evaluations and procedures in this study protocol including swallowing capsules without difficulty.

Ability to understand the purpose and risks of the study and provide signed and dated informed consent and authorization to use protected health information (in accordance with national and local subject privacy regulations).

Exclusion Criteria:

A life-threatening illness, medical condition or organ system dysfunction which, in the investigator's opinion, could compromise the subject's safety, interfere with the absorption or metabolism of Ibrutinib PO, or put the study outcomes at undue risk.

Any immunotherapy, chemotherapy, radiotherapy, or experimental therapy within 4 weeks before first dose of study drug (corticosteroids for disease-related symptoms allowed but require 1-week washout before study drug administration).

Central nervous system (CNS) involvement by lymphoma.

Major surgery within 4 weeks before first dose of study drug.

Creatinine >1.5 × institutional upper limit of normal (ULN); total bilirubin >1.5 × ULN (unless due to Gilbert's disease); and aspartate aminotransferase (AST) or alanine aminotransferase (ALT) >2.5 × ULN unless disease related.

Concomitant use of medicines known to cause QT prolongation or torsades de pointes.

Significant screening electrocardiogram (ECG) abnormalities including left bundle branch block, 2nd degree AV block type II, 3rd degree block, bradycardia, and QTc >470 msec.

Lactating or pregnant.

### Example 6: Safety and Efficacy of Combination of Ibrutinib and Ketoconazole in Subiects with Relapsed/Refractory Mantle Cell Lymphoma (MCL)

Purpose: The primary objective of this trial is to evaluate the efficacy of Ibrutinib in combination with ketoconazole in relapsed/refractory subjects with Mantle Cell Lymphoma (MCL). The secondary objective is to evaluate the safety of Ibrutinib in combination with ketoconazole in this population.

Primary Outcome Measures:

To measure the number of participants with a response to combination of Ibrutinib and ketoconazole.

Secondary Outcome Measures:

To measure the number of participants with adverse events as a measure of safety and tolerability.

To measure pharmacokinetics to assist in determining how the body responds to the study drug.

Patient reported outcomes (to measure the number of participants reported outcomes in determining the health related quality of life).

Eligibility:

18 Years and older; both genders are eligible.

Inclusion Criteria:

Men and women >18 years of age.

ECOG performance status of <2.

Pathologically confirmed MCL, with documentation of either overexpression of cyclin Dl or t(11;14), and measurable disease on cross sectional imaging that is >2 cm in the longest diameter and measurable in 2 perpendicular dimensions.

Documented failure to achieve at least partial response (PR) with, or documented disease progression disease after, the most recent treatment regimen.

At least 1, but no more than 5, prior treatment regimens for MCL (Note: Subjects having received >2 cycles of prior treatment with bortezomib, either as a single agent or as part of a combination therapy regimen, will be considered to be bortezomib-exposed.).

Willing and able to participate in all required evaluations and procedures in this study protocol including swallowing capsules without difficulty.

Ability to understand the purpose and risks of the study and provide signed and dated informed consent and authorization to use protected health information (in accordance with national and local subject privacy regulations).

Exclusion Criteria:

Prior chemotherapy within 3 weeks, nitrosoureas within 6 weeks, therapeutic anticancer antibodies within 4 weeks, radio- or toxin-immunoconjugates within 10 weeks, radiation therapy within 3 weeks, or major surgery within 2 weeks of first dose of study drug.

Any life-threatening illness, medical condition or organ system dysfunction which, in the investigator's opinion, could compromise the subject's safety, interfere with the absorption or metabolism of Ibrutinib capsules, or put the study outcomes at undue risk.

Clinically significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification.

Malabsorption syndrome, disease significantly affecting gastrointestinal function, or resection of the stomach or small bowel or ulcerative colitis, symptomatic inflammatory bowel disease, or partial or complete bowel obstruction.

Any of the following laboratory abnormalities: 1. Absolute neutrophil count (ANC) <750 cells/mm3 (0.75×109/L) unless there is documented bone marrow involvement. 2. Platelet count <50,000 cells/mm3 (50×109/L) independent of transfusion support unless there is documented bone marrow involvement. 3. Serum aspartate transaminase (AST/SGOT) or alanine transaminase (ALT/SGPT) >3.0 × upper limit of normal (ULN). 4. Creatinine >2.0 x ULN.

### Example 7: Phase 2 Study of the Combination of Ibrutinib and Ritonavir in High-Risk Chronic Lymphocytic Leukemia and Small Lymphocytic Lymphoma Patients

Purpose: The goal of this clinical research study is to learn if Ibrutinib combined with ritonavir can help to control chronic lymphocytic leukemia (CLL) and small lymphocytic lymphoma (SLL). The safety of this combination will also be studied.

Primary Outcome Measures:

Progression free survival (PFS) [Time Frame: 3 months]—progression free survival defined as the time interval from treatment to progressive disease or death, whichever happens earlier.

Patients in complete remission (CR), partial remission (PR) or stable disease (SD) are all counted as progression-free.

Survival or times to progression functions estimated using the Kaplan-Meier method.

Secondary Outcome Measures: Toxicity [Time Frame: 3 months]—toxicity reported by type, frequency and severity. Worst toxicity grades per patient tabulated for selected adverse events and laboratory measurements. Toxicity (grade 3 or 4) monitored based on the Bayesian model (beta-binomial) by assuming a priori probability of toxicity following beta(1,1).

Eligibility:

18 Years and older; both genders are eligible.

Inclusion Criteria:

Patients must have a diagnosis of high-risk CLL/SLL and be previously treated with up to 3 lines of prior therapy. High-risk CLL and high-risk SLL is defined by the presence of a 17p deletion or11q deletion or TP53 mutation. Any CLL and SLL patient who has a short remission duration of less than 3 years after prior first-line chemo-immunotherapy, such as the FCR regimen, also fulfills criteria of high-risk CLL/SLL, regardless of the presence or absence of cytogenetic abnormalities.

CLL and SLL patients with 17p deletion or TP53 mutation will not be required to have received any prior therapy, given the poor outcome of CLL/SLL patients to standard frontline chemo-immunotherapy, such patients will be eligible if they are untreated or if they have received up to 3 lines of prior therapy.

Patients must have an indication for treatment by 2008 IWCLL Criteria.

Patients age >18 years at the time of signing informed consent. Understand and voluntarily sign an informed consent. Be able to comply with study procedures and follow-up examinations.

ECOG/WHO performance status of 0-1.

Patients of childbearing potential must be willing to practice highly effective birth control (e.g., condoms, implants, injectables, combined oral contraceptives, some intrauterine devices [IUDs], sexual abstinence, or sterilized partner) during the study and for 30 days after the last dose of study drug. Women of childbearing potential include any female who has experienced menarche and who has not undergone successful surgical sterilization (hysterectomy, bilateral tubal ligation, or bilateral oophorectomy) or is not postmenopausal. Post menopause is defined as follows: Amenorrhea >/=12 consecutive months without another cause and a documented serum follicle stimulating hormone (FSH) level >35 mIU/mL; a male of childbearing potential is any male that has not been surgically sterilized.

Adequate renal and hepatic function as indicated by all of the following: Total bilirubin </=1.5 × institutional Upper Limit of Normal (ULN) except for patients with bilirubin elevation due to Gilbert's disease who will be allowed to participate; an ALT </1=2.5 ×ULN; and an estimated creatinine clearance (CrC1) of >30 mL/min, as calculated by the Cockroft-Gault equation unless disease related.

Free of prior malignancies for 3 years with exception of currently treated basal cell, squamous cell carcinoma of the skin, or carcinoma in situ of the cervix or breast.

A urine pregnancy test (within 7 days of Day 1) is required for women with childbearing potential

Exclusion Criteria:

Pregnant or breast-feeding females.

Treatment including chemotherapy, chemo-immunotherapy, monoclonal antibody therapy, radiotherapy, high-dose corticosteroid therapy (more than 60 mg Prednisone or equivalent daily), or immunotherapy within 21 days prior to enrollment or concurrent with this trial.

Investigational agent received within 30 days prior to the first dose of study drug or have previously taken Ibrutinib. If received any investigational agent prior to this time point, drug-related toxicities must have recovered to Grade 1 or less prior to first dose of study drug.

Systemic fungal, bacterial, viral, or other infection not controlled (defined as exhibiting ongoing signs/symptoms related to the infection and without improvement, despite appropriate antibiotics or other treatment).

Patients with uncontrolled Autoimmune Hemolytic Anemia (AIHA) or autoimmune thrombocytopenia (ITP).

Patients with severe hematopoietic insufficiency, as defined by an absolute neutrophil count of less than 500/micro-L and/or a platelet count of less than 30,000/micro-L at time of screening for this protocol.

Any other severe concurrent disease, or have a history of serious organ dysfunction or disease involving the heart, kidney, liver or other organ system that may place the patient at undue risk to undergo therapy with Ibrutinib and rituximab.

Significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification.

Significant screening ECG abnormalities including left bundle branch block, 2nd degree AV block type II, 3rd degree block, bradycardia, and QTc >470 msec.

Any serious medical condition, laboratory abnormality, or psychiatric illness that places the subject at unacceptable risk if he/she were to participate in the study.

History of stroke or cerebral hemorrhage within 6 months.

Evidence of bleeding diathesis or coagulopathy.

Major surgical procedure, open biopsy, or significant traumatic injury within 28 days prior to Day 1, anticipation of need for major surgical procedure during the course of the study. Minor surgical procedures, fine needle aspirations or core biopsies within 7 days prior to Day 1. Bone marrow aspiration and/or biopsy are allowed.

Serious, non-healing wound, ulcer, or bone fracture.

Any chemotherapy (e.g., bendamustine, cyclophosphamide, pentostatin, or fludarabine), immunotherapy (e.g., alemtuzumab, or ofatumumab), bone marrow transplant, experimental therapy, or radiotherapy is prohibited during therapy on this study.

Use of medications known to prolong QTc interval or that may be associated with Torsades de Pointes are prohibited within 7 days of starting study drug and during study-drug treatment.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

What is claimed is:

1. A pharmaceutical composition comprising:
a. a therapeutically-effective amount of Ibrutinib;
b. a CYP3A4 inhibitor; and
c. a pharmaceutically-acceptable excipient.

2. The pharmaceutical composition of claim 1, wherein the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof.

3. The pharmaceutical composition of claim 1, wherein the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof.

4. The pharmaceutical composition of claim 3, wherein the CYP3A4 inhibitor is cobicistat (GS-9350) or analogs or derivatives of cobicistat (GS-9350).

5. The pharmaceutical composition of claim 3, wherein the CYP3A4 inhibitor is ketoconazole.

6. The pharmaceutical composition of claim 3, wherein the CYP3A4 inhibitor is ritonavir.

7. The pharmaceutical composition of claim 1, wherein the therapeutically-effective amount of Ibrutinib is between about 10 mg to about 100 mg.

8. The pharmaceutical composition of claim 7, wherein the therapeutically-effective amount of Ibrutinib is between about 40 mg and about 100 mg, or about 40 mg and about 70 mg.

9. The pharmaceutical composition of claim 7, wherein the therapeutically-effective amount of Ibrutinib is about 40 mg.

10. The pharmaceutical composition of claim 1, in a combined dosage form.

11. A method of treating a B-cell proliferative disorder in an individual in need thereof comprising administering a combination of:
a. a therapeutically-effective amount Ibrutinib; and
b. a CYP3A4 inhibitor.

12. The method of claim 11, wherein the B-cell proliferative disorder is chronic lymphocytic leukemia (CLL), small lymphocytic lymphoma (SLL), high risk CLL, or a non-CLL/SLL lymphoma.

13. The method of claim 11, wherein the B-cell proliferative disorder is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma.

14. The method of claim 11, wherein the B-cell proliferative disorder is acute or chronic myelogenous (or myeloid) leukemia, myelodysplastic syndrome, or acute lymphoblastic leukemia.

15. The method of claim 11, wherein the B-cell proliferative disorder is relapsed or refractory diffuse large B-cell lymphoma (DLBCL), relapsed or refractory mantle cell lymphoma, relapsed or refractory follicular lymphoma, relapsed or refractory CLL;
relapsed or refractory SLL; relapsed or refractory multiple myeloma.

16. The method of claim 11, wherein the B-cell proliferative disorder is high risk CLL or high risk SLL.

17. The method of claim 11, wherein the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof.

18. The method of claim 11, wherein the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin; verapamil; telaprevir; vincristine; voriconazole; or any combinations thereof.

19. The method of claim 18, wherein the CYP3A4 inhibitor is cobicistat (GS-9350) or analogs or derivatives of cobicistat (GS-9350).

20. The method of claim 18, wherein the CYP3A4 inhibitor is ketoconazole.

21. The method of claim 18, wherein the CYP3A4 inhibitor is ritonavir.

22. The method of claim 11, wherein the therapeutically-effective amount of Ibrutinib is between about 10 mg to about 100 mg.

23. The method of claim 22, wherein the therapeutically-effective amount of Ibrutinib is between about 40 mg and about 100 mg, or about 40 mg and about 70 mg.

24. The method of claim 22, the therapeutically-effective amount of Ibrutinib is about 40 mg.

25. The method of claim 11, wherein Ibrutinib and the CYP3A4 inhibitor are in a combined dosage form.

26. The method of claim 11, wherein Ibrutinib and the CYP3A4 inhibitor are in separate dosage forms.

27. The method of claim 11, wherein Ibrutinib and the CYP3A4 inhibitor are administered concurrently.

28. The method of claim 11, wherein Ibrutinib and the CYP3A4 inhibitor are administered simultaneously, essentially simultaneously or within the same treatment protocol.

29. The method of claim 11, wherein Ibrutinib and the CYP3A4 inhibitor are administered sequentially.
